(19) United States
(12) Patent Application Publication
(10) Pub. No.: US 2006/0235207 A1
(43) Pub. Date: Oct. 19, 2006

(54) Antibodies against lesion tissue

(75) Inventors: Masayuki Tsuchiya, Shizuoka (JP); Masami Suzuki, Shizuoka (JP); Kenji Yoshida, Shizuoka (JP); Etsuko Fuji, Shizuoka (JP); Kouichi Matsubara, Singapore (SG); Hiroyuki Tsunoda, Shizuoka (JP)

Correspondence Address: FISH & RICHARDSON PC, P.O. BOX 1022, MINNEAPOLIS, MN, 55440-1022, US

(73) Assignees: CHUGAI SEIYAKU KABUSHIKI KAISHA, Tokyo (JP); PHARMALOGICALS RESEARCH PTE. LTD., Singapore (SG)

(21) Appl. No.: 10/535,764

(22) PCT Filed: Nov. 21, 2003
(86) PCT No.: PCT/JP03/14919

§ 371 Date: Mar. 15, 2006

(30) Foreign Application Priority Data
Nov. 22, 2002 (JP) 2002-339241

(51) Int. Cl. C07K 16/30; G01N 33/574; C07H 21/04; C12P 21/06; C12N 5/06

(52) U.S. Cl. 530/388.8; 435/7.23; 435/69.1; 435/338; 435/320.1; 536/23.53

(57) ABSTRACT

Methods for isolating polynucleotides encoding antibodies against lesional tissues are provided, wherein the methods comprise the steps of: (a) isolating a B cell(s) that infiltrates into a lesional tissue of interest; and (b) obtaining an antibody-encoding polynucleotide from the isolated B cell(s). The lesions may be a cancer tissue or such. Antibody genes can be obtained without depending on B cell cloning. Accordingly, it is also possible to obtain genes encoding human-derived antibodies which are difficult to clone. Genes that encode antibodies against cancer can be obtained using cancer tissues as the lesion.

### TECHNICAL FIELD

The present invention relates to antibodies against lesional tissues and methods for producing the same.

### BACKGROUND ART

Lymphocytes are widely known to infiltrate cancer tissues (Document 1/ Hurliamnn et al. (1985) Int J Cancer 35:753; Document 2/ Whiteside et al. (1986) Cancer Immunol Immunother 23:169; Document 3/ Wolf et al. (1986) Otolaryngol Head Neck Surg 95:142; Document 4/ Husby et al. (1976) J Clin Invest 57:1471; Document 5/ Vose et al. (1979) Int J Cancer 24:579). Experimental and clinical data suggest that lymphocyte infiltration of cancer tissues is associated with host immunoreactions against cancer (Document 5/ Rosenberg et al. (1988) New Engl J Med 319:1676; Document 6/ Van Pel et al. (1995) Immunol Reviews 145:229; Document 7/ Kreider et al. (1984) Cancer Metastasis Rev 3:53).

In the host immune defense system against cancer, cytotoxic T cells (CTLs) are the effector cells that directly kill cancer cells (Document 8/ Nobholz and MacDonald (1983) Annu Rev Immunol 1:273). Furthermore, some of the antibodies produced by plasma cells, which are the final form of differentiated B cells, are assumed to have the ability to bind to cancer cells (Document 9/ Roitt et al. (1969) Lancet 2: 367; Document 10/ Borsos (1971) Progress in Immunology: p841. New York, Academic Press; Document 11/ Kodera and Bean (1975) Int J Cancer 16:579).

For example, it has been shown that B cells which infiltrate cancer tissues express antibodies, and that these antibodies bind selectively to antigens on cancer cells (Document 12/ Punt et al. (1994) Cancer Immunol Immunother 38:225; Document 13/ Zhang et al. (1995) Cancer Res 55:3584). This suggests that cancer antigens can be identified by using antibodies expressed in infiltrating B cells. Antibodies having such reactivity will be useful for cancer treatment and diagnosis if they can be obtained.

Antibodies that bind to cancer cells destroy cancer cells by activating the complement system or by triggering antibody-dependent cytotoxicity. However, in reality, there have been few reports on the specificity and the repertoire of variable regions in antibodies produced by cancer tissue-infiltrating B cells.

One of the reasons why it is difficult to analyze antibodies expressed in cancer tissue-infiltrating B cells is the difficulty of isolating antibodies that are produced by infiltrating B cells. Antibody analysis generally requires the cloning of antibody-producing cells. As a technique for cloning antibody-producing cells, a method that immortalizes human B cells using Epstein-Barr viruses (EBVs) is known. However, the probability of establishing a human antibody-producing cell line through EBV infection is extremely low (Document 14/ Henderson et al. (1977) Virology 76:152; Document 15/Aman et al. (1984) J Exp Med 159:208).

The hybridoma method devised for establishing murine antibody-producing cell lines is another technique for cloning antibody-producing cells. The hybridoma method comprises the step of fusing an antigen-specific B cell with an immortalized myeloma cell to establish an antibody-expressing cell line. However, no partner cells that efficiently fuse with human B cells have been found so far. Murine myeloma cells are effective in the case of murine hybridomas; however, when used as a fusion partner for human B cells, characteristics of human hybridomas become unstable due to the preferential occurrence of deletions in human chromosomes, thus failing to lead to the establishment of antibody-producing cell lines (Document 16/ Winter and Milstein (1991) Nature 349:293). At present, it is thus technically difficult to establish human-derived antibody-producing cell lines.

Recombinant DNA techniques for producing antibodies with similar antigen-binding activity as the cloned antibody-producing cells, that is, methods for cloning antibody genes and preparing recombinant antibody proteins, have been established (Document 17/ Marks et al. (1991) J Mol Biol 222:581; Document 18/ Larrick et a/ (1992) Immunol Reviews 130:69). Antibody genes encoding, for example, Fv, scFv, Fab, IgQ or IgM, can be generated by cloning genes that encode antibody variable regions (Document 19/ Skerra et al. (1988) Science 240:293; Document 20/ Bird et al. (1988) Science 242:423; Document 21/ Better et al. (1988) Science 240:1041). The smallest recombinant antibody molecule, scFv, has a structure in which the heavy chain variable region and the light chain variable region are connected through a linker.

Needless to say, cloned B cells express a single antibody gene. Accordingly, antibodies having an activity similar to that of antibodies produced by B cells can be reconstructed by cloning the heavy and light chain variable regions from these cells. However, B cells present in peripheral blood and those infiltrating cancer tissues are (polyclonal) cell populations which produce various antibodies (Document 22/ Kotlan et al. (1999) Immunol Lett 65:143; Document 23/Hansen et al. (2001) Pro Natl Acad Sci USA 98:12659). Therefore, it would not be easy to clone antibody genes from such cell populations and reconstruct them as human antibodies.

### DISCLOSURE OF THE INVENTION

An objective of the present invention is to provide methods for obtaining polynucleotides encoding antibodies having a specific reactivity from antibody-producing cells comprised in a polyclonal cell population.

In general, to clone antibody genes using gene recombination techniques, B cells cloned by some technique are required. This limitation poses difficulties for, for example, obtaining antibodies produced by B cells which have infiltrated cancer tissues. The present inventors searched for methods that would enable one to obtain antibody genes without having to rely on B cell cloning, and conceived that it may be possible to utilize cellular mRNAs isolated from cell populations as a cloning source. In general, B cells in peripheral blood are used to clone human antibody genes. However, since B cell populations in peripheral blood are polyclonal cell populations producing antibodies with various reactivities, these B cells were thought to be inappropriate for selectively isolating antibodies with a specific reactivity.

Microdissection is a technique for excising and isolating specific cells from a specimen composed of heterogeneous cell populations such as a tissue section. For example, a system for isolating cells in which the periphery of target cells is excised with an ultraviolet laser has been put into practical use. This system is referred to as laser microdissection (LMD), and is commercially available. LMD has spread as a technique that can be used to obtain target cells with high precision and little damage to cells. By using LMD, a specific cellular gene can be obtained and amplified by PCR.

The present inventors thought that B cells which have infiltrated cancer tissues are very likely to produce antibodies having reactivities to cancer cells, and therefore, polynucleotides encoding antibodies which bind to cancer cells may be efficiently isolated by isolating infiltrating B cells using the LMD system or such. Then, the present inventors showed that polynucleotides encoding the antibodies can be obtained by utilizing, as a cloning source, B cells which have actually infiltrated a lesional tissue, and thereby completed the present invention. That is, the present invention, as described below, relates to methods for isolating antibody genes and preparing antibodies encoded by these genes, and antibodies obtained by these methods.

[1] A method for isolating a polynucleotide encoding an antibody against a lesional tissue, wherein the method comprises the steps of:
(a) isolating a B cell infiltrating into a lesional tissue; and
(b) obtaining a polynucleotide encoding an antibody from the isolated B cell.

[2] The method of [1], wherein the lesional tissue is a cancer tissue.

[3] The method of [1], wherein the step (a) of isolating a B cell infiltrating into a lesional tissue comprises the step of excising a region comprising the B cell from a section of the lesional tissue.

[4] The method of [1], wherein the step (b) of obtaining a polynucleotide encoding an antibody comprises the step of amplifying a gene encoding an antibody variable region.

[5] A polynucleotide, which is isolated by the method of [1] and which encodes an antibody.

[6] The polynucleotide of [5], wherein the polynucleotide encoding an antibody comprises a polynucleotide encoding an antibody variable region.

[7] An expression vector comprising the polynucleotide of [5].

[8] A host cell comprising the polynucleotide of [5] or the expression vector of [7].

[9] A method for manufacturing an antibody, wherein the method comprises the steps of:
(1) culturing the host cell of [8]; and
(2) recovering an antibody as an expression product.

[10] An antibody which is manufactured by the method of [9].

[11] An antibody which is encoded by the polynucleotide of [5].

[12] The method for manufacturing the antibody of [9], wherein the method further comprises the steps of:
(1) contacting the antibody obtained by the method of [9] with a lesional tissue;
(2) detecting the binding of the antibody to said lesional tissue; and
(3) selecting the antibody binding to said lesional tissue.

The present invention relates to methods for isolating a polynucleotide encoding an antibody against a lesion, wherein the methods comprise the steps of:
(a) isolating a lesion-infiltrating B cell; and
(b) obtaining an antibody-encoding polynucleotide from the isolated B cell.

B cells which have infiltrated a lesion are very likely to produce antibodies against that lesion. In other words, it can be said that lesions have accumulations of B cells that produce antibodies recognizing the lesion. Therefore, antibody genes derived from B cells isolated from a cell population that has infiltrated a lesion is useful for generating antibodies against that lesion. In this invention, the term “antibodies against a lesion” refers to antibodies that recognize the antigens which constitute the lesion or antigenic substances produced at the lesion. Such antibodies are useful for diagnosing and treating the lesion. Furthermore, when the lesion is due to an autoimmune disease, such antibodies provide important information for epitope analysis in the autoimmune disease.

For the methods of the present invention, any lesion recognized as non-self by the immune system can be utilized. For example, the following lesions are preferable for obtaining B cells. These lesions can be referred to as naturally occurring lesions, which can also be obtained from human subjects being treated for said lesions.

solid tumor lesions

arteriosclerosis lesions

inflammatory disease lesions

lesions generated by infectious pathogens

autoimmune disease lesions

On the other hand, artificially prepared lesions can also be utilized in this invention. For example, the following lesions are artificially prepared lesions, and can be obtained from, for example, immune animals. By utilizing artificial lesions, polynucleotides encoding antibodies against any antigen can be obtained.

heterogenous cells and tissues artificially transplanted into immune animals

cells and tissues that express non-self genes artificially transplanted into immune animals

Cancer tissues can be exemplified as preferable lesions of the present invention. Specifically, the present invention relates to methods for isolating a polynucleotide encoding an antibody against a cancer cell, wherein the method comprises the steps of:
(a) isolating a cancer tissue-infiltrating B cell; and
(b) obtaining an antibody-encoding polynucleotide from the isolated B cell.

Cancer tissues to be used in this invention are not limited. Specifically, breast cancer tissues, lung cancer tissues, liver cancer tissues, colon cancer tissues, pancreas cancer tissues, prostate cancer tissues, and such can be used. Among them, cancers comprising many B cell infiltrations are preferable cancer tissues of this invention. Examples of cancers comprising many B cell infiltrations include breast cancers, lung cancers, and melanomas. Cancer tissues can be removed by surgical excision. For example, cancer tissues removed for biopsy can be used as cancer tissues for the present invention. Furthermore, tissues taken out from patients by surgical removal are also useful as cancer tissues. These tissues can be those removed for obtaining antibody genes; alternatively, tissues removed for histopathological examination or surgical treatment can also be used.

In this invention, any method may be used for isolating B cells which infiltrate cancer tissues. The preferable methods for isolating B cells include microdissection, a technique for excising specific cells from a tissue section. For example, target cells can be isolated from frozen tissue sections using the Laser Microdissection (LMD) system. A system for excising tissue sections with an ultraviolet laser is commercially available. The use of this system, which involves specifying the region to be excised on a computer image under microscopic observation, makes it possible to excise any region from a tissue section.

In this case, a large number of B cells can be isolated by observing a specimen under a microscope and selecting a portion enriched with B cells. Alternatively, a small number of B cells can be easily obtained by excising a region with low B cell density. As shown in the Examples, it is even possible to obtain a single cell.

In the present invention, B cells can be isolated from any specimen prepared for pathological analysis. For example, thin-section frozen specimens are preferable pathological specimens in this invention. As a pathological specimen, not only fresh tissues but also specimens fixed with paraformaldehyde (PFA) or such can be used. Accordingly, it is also possible to obtain antibody genes from, for example, preserved pathological specimens using the methods of the present invention. As described above, a broad range of cloning sources can be selected for the methods of this invention. That is, the present invention provides methods that can be conveniently used to obtain a variety of antibody genes.

Microdissection is a system also utilized in gene analysis of specialized cells in tissues using PCR or such. However, there has been no report on the utilization of microdissection for obtaining antibody genes. The present inventors focused on the fact that it is possible to use lesion-infiltrating B cell populations as cell populations that are very likely to produce antibodies with a desired reactivity. Furthermore, by using the antibody-producing cells obtained from such a cell population as the cloning source, the present inventors thus made it possible to obtain antibody genes.

More specifically, B cells or plasma cells which infiltrate cancer tissues and produce antibodies can be extracted based on pathological analysis under microscopic observation. B cells and plasma cells can be distinguished by staining with toluidine blue or such. Compared with conventional methods in which B cells or plasma cells are extracted from tissue fractions comprising mixtures of peripheral blood and cancerous/non-cancerous portions, this method makes it possible to isolate genes of antibodies that recognize cancer cells with a much higher probability.

In the present invention, the term “cell isolation” refers to the separation of B cells from a cell population comprising mixtures of heterogeneous cells. The “cell isolation” of the present invention includes isolation of cells harboring antigen genes when they coexist with other cells that do not carry an antibody gene. For example, as shown in the Examples, carrier cells that are clearly incapable of producing antibodies may be added to the antigen-producing cells. Carrier cells are added to facilitate the extraction of target mRNAs. That is, even if only a limited number of antibody-producing cells are coexisting with other cells which do not carry antibody genes, these antibody-producing cells can be said to be in an isolated state.

In this invention, the number of B cells to be isolated is not limited. Specifically, for example, one to 100 cells, usually one to 50 cells, preferably 20 cells or less, more preferably five cells or less, and yet more preferably one cell is isolated.

Isolation of single cells enhances the possibility of obtaining an antibody gene in a state that maintains its heavy and light chain combinations. When reconstructing functional antibody molecules, it is an important condition to obtain an antibody gene in such a state. In cloning antibody genes, the use of monoclonal antibody-producing cells as a cloning source is an essential condition for accurately reconstructing combinations of heavy and light chains. However, if an antibody gene obtained from a single cell is used, repeated cloning of the gene would be required when comparing multiple antibodies with different reactivities. Comparison of multiple antibodies is useful for obtaining a suitable antibody with purpose-oriented characteristics.

On the contrary, when multiple cells are isolated as the cloning source, combinations of the heavy and light chains of the obtained antibody genes cannot be identified. However, it is possible to obtain antibody genes derived from multiple cells, that is, a library of antibody genes. By screening such a library with antibody activity as an index, genes of antibodies with the desired reactivity can be obtained. It is impossible to confirm whether heavy and light chain combinations of an antibody gene selected by screening are derived from the same cell. However, regardless of whether the genes are derived from the same cell or not, the purpose of screening is accomplished by obtaining antibodies having the desired reactivity.

In the methods of the present invention, any lesion-infiltrating antibody-producing cell can be used as a cloning source. Normally, B cells circulating in peripheral blood have various levels of differentiation. At the early stages of differentiation, B-cells have L-chains on their surfaces as an antigen receptor. B cells mature and differentiate into IgG-secreting cells through differentiation and activation by antigen stimulation. B cells at the final stage of differentiation are referred to as plasma cells (or plasmocytes). Plasma cells produce 2,000 molecules of IgG per second. Therefore, by isolating plasma cells, more mRNAs can be obtained.

In general, many lesion-infiltrating B cells are found to be at an advanced stage of differentiation. Further, it is highly likely that the infiltrating B cells produce antibodies against the lesion. Therefore, by using B cells isolated from a lesion as the cloning source, the specificity of the resulting antibody will be naturally centered on the lesion. Furthermore, antibody genes are expressed at an extremely high level in plasma cells and in B cells at an advanced stage of differentiation. Accordingly, utilization of such cells as the cloning source leads to increased possibility of obtaining antibody genes. By utilizing lesion-infiltrating B cells, antibody genes of interest can be obtained with a high probability using relatively few cells.

Antibody genes can be obtained from isolated B cells by amplifying the antibody genes. Methods for amplifying genes are known in the art. For example, the PCR method is preferred as a method for amplifying antibody genes. A method for isolating antibody genes using the PCR method is described below.

First, mRNAs are extracted from isolated B cells. cDNAs are synthesized by using the extracted mRNAs as a template to obtain a cDNA library. Commercially available kits are conveniently used for extracting mRNAs and for constructing the cDNA library. In this invention, mRNAs derived from a small number of B cells are used. In practice, mRNAs obtained from only few cells are extremely small in amount, and have low yields when directly purified. Therefore, mRNAs are usually purified after the addition of carrier RNAs that clearly contain no antibody genes. Alternatively, with a certain amount of RNAs being extracted, the RNAs of antibody-producing cells themselves can also be efficiently extracted. The addition of carrier RNAs may not be required for extracting RNAs from, for example, 10 or more, 30 or more, preferably 50 or more antibody-producing cells.

By using the cDNA library thus obtained as a template, antibody genes can be amplified by the PCR method. Primers for amplifying antibody genes by the PCR method are known in the art. For example, primers for amplifying human antibody genes can be designed based on the disclosures in research papers (J. Mol. Biol. (1991) 222, 581-597) and web sites (http://www.mrc-cpe.cam.ac.uk/vbase-ok.php?menu=901). These primers have different nucleotide sequences depending on the subclass of immunoglobulins. Accordingly, when a cDNA library with unknown subclasses is used as a template, the PCR method is performed considering all possibilities.

For example, to obtain genes encoding human IgQ primers that allow the amplification of genes encoding γ1 to γ5 as the heavy chain, and κ-chain and λ-chain as the light chains, can be used. For amplifying genes of the IgG variable region, a primer capable of annealing to a portion that corresponds to the hinge region is generally used as the 3′ primer. Meanwhile, a primer corresponding to each subclass can be used as the 5′ primer.

PCR products obtained using primers for amplifying genes of the respective heavy and light chain subclasses are considered as independent libraries. Using thus synthesized libraries, it is possible to reconstitute immunoglobulins comprising combinations of heavy chains and light chains. The lesion-binding activity of the reconstituted immunoglobulin can be used as an index to screen for antibodies of interest.

For example, to obtain antibodies targeting cancer tissues, it is preferred that the antibodies of the present invention bind to cancer cells. It is even more preferable that the antibodies bind specifically to cancer cells. Cancer-binding antibodies can be screened by, for example, the following steps of:
(1) contacting an antibody obtained by a method of the present invention with a cancer cell;
(2) detecting binding between the antibody and the cancer cell; and
(3) selecting an antibody that binds to the cancer cell.

Methods for detecting binding between antibodies and cancer cells are known in the art Specifically, an immobilized cancer specimen is reacted with a test antibody, and then with a labeled secondary antibody which recognizes the test antibody. The binding of the test antibody to cancer cells can be confirmed by detecting labeled antibody on the immobilized specimen after washing. Enzymatically active proteins such as peroxidase and β-galactosidase, and fluorescent substances such as FITC can be used as the label. Cancer tissues to be analyzed for antibody-binding activity may be cancer tissues forming the lesion from which B cells are obtained. Alternatively, cancer tissues of the same organ which have been removed from different individuals, and cell lines derived from these cancer tissues, can be also used. Furthermore, cancer tissues of different organs, or cell lines derived therefrom, can also be used to screen for antibodies which commonly react with different types of cancers.

In the present invention, an antibody whose reactivity towards a cancer tissue is significantly high compared to the normal tissue can be said to bind specifically to the cancer. For comparing the reactivities of the antibodies of this invention, the same type of tissue is generally used. That is, the reactivity of an antibody is compared between a cancer tissue and a normal tissue of the organ from which the cancer tissue is derived. Under conditions in which an antibody's reactivity towards a cancer tissue can be confirmed, the antibody is said to have a specific reactivity towards the cancer tissue when no binding activity towards the normal tissue can be detected.

Methods of screening for antibodies using binding activity as an index include the panning method which utilizes phage vectors. When the obtained antibody genes are gene libraries of heavy and light chain subclasses as described above, screening methods that use phage vectors are advantageous. As described in the Examples, genes encoding variable regions of the heavy and light chains can be made into single chain Fv (scFv) through conjugation with a suitable linker sequence. Phages expressing scFv on their surface can be obtained by inserting a scFv-encoding gene into a phage vector. By contacting these phages with an antigen of interest and recovering the phages bound to the antigen, DNAs encoding scFv with the desired binding activity can be recovered. scFv having the desired binding activity can be concentrated by repeating this procedure as necessary.

An antibody-encoding polynucleotide of the present invention may encode a whole antibody or a portion of the antibody. The term “a portion of the antibody” means any portion of an antibody molecule. Hereinbelow, a portion of an antibody may be referred to as “an antibody fragment”. Preferred antibody fragments of the present invention comprise the complementarity determination region (CDR) of an antibody. More preferably, antibody fragments of the present invention comprise all three CDRs that constitute the variable region.

For example, polynucleotides encoding the variable region of an antibody are preferred antibody fragments of the present invention. A complete immunoglobulin molecule can be reconstituted by obtaining a polynucleotide encoding a variable region and linking the polynucleotide with a polynucleotide encoding a constant region. Constant regions of antibodies within the same class have approximately the same structure. That is, the structure of the constant region has no effect on the antigen-binding activity. Accordingly, if the structure of the variable region of an antibody can be elucidated, antibodies having similar activity can be reconstituted through conjugation with the constant region that is already obtained.

Antibodies of the present invention include genetically recombinant antibodies whose structures have been artificially modified. For example, mouse-human chimeric antibodies can be prepared by: conjugating a human constant region gene with an antibody gene obtained not from a human but from a non-human animal such as a mouse. Methods for humanizing a mouse variable region by transferring CDRs which constitute the variable region of a non-human animal such as mouse to a human variable region are also known in the art. Polynucleotides encoding the antibodies of the present invention can be DNAs, RNAs, or chimeric molecules thereof. Furthermore, artificial structures such as PNAs can also be included, as long as the nucleotide sequence is maintained. Methods for synthesizing polynucleotides having the same nucleotide sequence, based on the nucleotide sequence of a gene that encodes an antibody isolated from B cells, are known in the art.

The polynucleotides of the present invention can comprise a sequence identical or highly homologous to the gene encoding an antibody isolated from B cells. Herein, the term “highly homologous” generally refers to a homology of 70% or more, preferably 80% or more, more preferably 90% or more, and even more preferably 95% or more.

The present invention relates to polynucleotides encoding antibodies obtained by the above-described methods. The polynucleotides of this invention can be incorporated into any expression vector. Appropriate host cells can be transformed with the expression vector to obtain antibody-expressing cells. By culturing the antibody-expressing cells and then recovering expression products therefrom, antibodies encoded by the genes can be obtained. The following describes expressing antibody genes isolated by the above-described methods.

When the antibody genes have been isolated and introduced into an appropriate host, hosts and expression vectors can be used in appropriate combinations to produce the antibodies. As eukaryotic host cells, animal cells, plant cells, and fungal cells may be used. Known animal cells include: (1) mammalian cells such as CHO, COS, myeloma, baby hamster kidney (BHK), HeLa, and Vero cells; (2) amphibian cells such as Xenopus oocytes; or (3) insect cells such as sf9, sf21, and Tn5. Known plant cells include cells derived from the Nicotiana genus such as Nicotiana tabacum, which can be callus cultured. Known fungal cells include yeasts such as the Saccharomyces genus, for example Saccharomyces cerevisiae, and filamentous fungi such as the Aspergillus genus, for example Aspergillus niger. Prokaryotic cells can also be used in production systems that utilize bacterial cells. Known bacterial cells include E. coli and Bacillus subtilis. The antibodies can be obtained by transferring the antibody genes of interest into these cells using transformation, and then culturing the transformed cells in vitro.

Furthermore, the antibodies obtained by the methods of the present invention may be antibody fragments or modified antibodies thereof. For example, the antibody fragments may be Fab, F(ab′)2, Fv, or single chain Fv (scFv) in which Fv from H or L chains are ligated by an appropriate linker. More specifically, the antibody fragments can be obtained by treating the antibodies with enzymes such as papain and pepsin to produce antibody fragments. Alternatively, genes encoding these antibody fragments can be prepared and introduced into expression vectors for expression in appropriate host cells (see, for example, Co, M. S. et al., J. Immunol. (1994) 152, 2968-2976; Better, M. & Horwitz, A. H. Methods in Enzymology (1989) 178, 476496, Academic Press, Inc.; Plueckthun, A. & Skerra, A. Methods in Enzymology (1989) 178, 476-496, Academic Press, Inc.; Lamoyi, E., Methods in Enzymology (1989) 121, 663-669; and Bird, R- E. et al., TIBTECH (1991) 9, 132-137).

scFv can be obtained by ligating the V regions of the antibody H-chain and L-chain. In the scFv, the V regions of the H chain and L chain are ligated via a linker, and preferably via a peptide linker (Huston, J. S. et al., Proc. Natl. Acad. Sci. U.S.A (1988) 85, 5879-5883). The V regions of the scFv H chain and L chain may be derived from any of the antibodies described herein.

The peptide linker used to ligate the V regions may be any single-chain peptide consisting of 12 to 19 residues. DNA encoding scFv can be amplified by PCR using as a template either the whole DNA, or a partial DNA encoding a desired DNA, selected from a DNA encoding the H chain or the V region of the H chain of the above antibody, and a DNA encoding the L chain or the V region of the L chain of the above antibody; and using a primer pair that defines the two ends. Further amplification can be subsequently conducted using the combination of DNA encoding the peptide linker portion, and the primer pair that defines both ends of the DNA to be ligated to the H chain and the L chain respectively. Once DNAs encoding scFvs are constructed, expression vectors containing the DNAs, and hosts transformed by these expression vectors, can be obtained according to conventional methods. Furthermore, scFvs can be obtained according to conventional methods using the resulting hosts.

These antibody fragments can be produced in hosts by obtaining genes encoding the antibody fragments and expressing them in a similar manner to that outlined above. Antibodies bound to various types of molecules, such as polyethylene glycol (PEG), may be used as modified antibodies. Furthermore, antibodies may bind to radioisotopes, chemotherapeutics, cytotoxic substances such as bacteria-derived toxin, and labeling substances. Such modified antibodies can be obtained by chemically modifying the resulting antibodies. Methods for modifying antibodies are already established in the art. The term “antibody” in the present invention also encompasses the modified antibodies as described above.

Furthermore, the antibodies of the present invention may be bispecific antibodies. The bispecific antibodies may have antigen-binding sites recognizing different epitopes on the antigen molecules, or may have one antigen-binding site recognizing antigens and the other recognizing a cytotoxic substance such as radioactive substance, chemotherapeutic agent, and cell-derived toxin. In this case, it is possible to inhibit the growth of cancer cells by directly applying the cytotoxic substance to the cells expressing antigens to specifically damage them. Bispecific antibodies can be prepared by linking HL pairs of two kinds of antibodies, or obtained by fusing hybridomas that produce different monoclonal antibodies to prepare fused cells generating bispecific antibodies. Furthermore, bispecific antibodies can be generated by using genetic engineering techniques.

Antibodies expressed and produced as described above can be purified by conventional methods for purifying normal proteins. Antibodies can be separated and purified by, appropriately selecting and/or combining affinity columns such as protein A columns or chromatography columns, filtration, ultrafiltration, salt precipitation, dialysis, and such (Antibodies A Laboratory Manual. Ed Harlow, David Lane, Cold Spring Harbor Laboratory, 1988).

Conventional means can be used to measure the antigen-binding activity of the antibodies (Antibodies A Laboratory Manual. Ed Harlow, David Lane, Cold Spring Harbor Laboratory, 1988). For example, enzyme linked immunosorbent assays (ELISAs), enzyme immunoassays (EIAs), radioimmunoassays (RIAs), or fluoroimmunoassays may be used.

Techniques common to the field of genetic engineering can be used to carry out procedures for constructing an expression system for producing the antibodies of the present invention, and for constructing recombinant vectors appropriate for the hosts (for example, Sambrook et al., Molecular Cloning, Cold Spring Harbor Laboratories (1989)). Host cells may be prokaryotic cells such as bacteria, and eukaryotic cells such as yeast, animal cells, insect cells, and plant cells, as long as the cells are capable of expressing the light chains of the present invention or the antibodies comprising the light chains. Mammalian cells are particularly preferred in view of glycosylation.

Expression vectors need to comprise units that regulate the transcription and translation of genetic information, such as promoters and terminators. For example, when Escherichia microorganisms such as E. coli are used as hosts, plasmids of the pBR or pUC series can be used as plasmid vectors, and any promoters selected from those such as lac, trp, tac, trc, λ phage PL, and PR can be used. Terminators may originate from trpA, phage, and rrnB ribosomal RNA.

When the hosts are Bacillus microorganisms such as B. subtilis, plasmids such as those of the pUB110 and pC194 series can be used, and genes may be integrated into chromosomes in some cases. Promoters and terminators may be derived from apr, npr, amy, and such.

Other prokaryotic cells include microorganisms such as Pseudomonas (e.g. P. putida, P. cepacia; pKT240 vectors, and such), Brevibacteria (e.g. B. lactofermentum; pAJ43), Corynebacteria (e.g. C. glutamicum; pCS11, pCB101), Streptococcus (e.g. pHV1301, pGK1), Lactobatcillaceae (e.g. pAMβ1), Rhodcoccus (e.g. plasmids isolated from R. rhodochrous (J. Gen. Microbiol. 138: 1003 (1992)), Streptomyces (e.g. S. lividans, S.virginiae; pIJ486, pKC1064, pUWL-KS), Enterobacter, Erwinia, Klebsiella, Proteus, Salmonella (e.g. S. typhimurium), Serratia (e.g. S. marcescans), and Shigella.

Among expression systems utilizing eukaryotic microorganisms, a system using Saccharomyces cerevisiae as a host, and plasmids from YRp, YEp, YCp, and YIp series is known. Therein, promoters and terminators such as ADH, GAPDH, PHO, GAL, PGK, and ENO can be used. Other microorganisms used in the expression vector systems of the present invention include Kluyveromyces (e.g. K. lactis; plasmids of the 2 μm, pKD1, pGKI1, and KARS series, and such), Schizosaccharomyces (e.g. S. pombe; pAUR224), Zygosaccharomyces (e.g. Z. rouxii; pSB3 and PH05 promoters from S. cerevisiae), Hansenula (e.g. H. polymorpha), Pichia (e.g. P. pastoris), Candida (e.g. C. maltosa, C. tropicalis, C. utilis, and C. albicans), Aspergillus (e.g. A. oryzae, A. niger), and Trichoderma (e.g. T. reesei).

In another embodiment, plant cells may be used as hosts. For example, host cells may be those from cotton, corn, potato, tomato, soybean, petunia, and tobacco. A particularly well-known system uses cells from Nicotina tabacum, which are cultured as a callus. To transform plant cells, expression vectors such as pMON530 are introduced into bacteria such as Agrobacterium tumefaciens. By infecting these bacteria into tobacco (Nicotina tabacum), desired polypeptides can be obtained from the tobacco leaves.

Cells from insects such as silkworms (Bombyx mori), mosquitoes (e.g. Aede aegypti, Aedes albopictus) and fruit flies (Drosophila melanogaster) can be used as hosts. For example, when using silkworms as hosts, DNAs encoding antibodies may be inserted into baculovirus vectors, these vectors may be used to infect silkworms, and desired polypeptides can be obtained from the silkworm body fluids (Nature 315: 592-594 (1985)).

Examples of expression vectors when using animal cells as hosts include pME18S (Med. Immunol. 20: 27-32 (1990)), pEF-BOS (Nucleic Acids Res. 18: 5322 (1990)), pCDM8 (Nature 329: 840-842 (1987)), pRSVneo, pSV2-neo, pcDNAI/Amp (Invitrogen), pcDNAI, pAMoERC3Sc, pCDM8 (Nature 329: 840 (1987)), pAGE107 (Cytotechnology 3: 133 (1990)), pREP4 (Invitrogen), pAGE103 (J. Biochem. 101: 1307 (1987)), pAMoA, pAS3-3, pCAGGS (Gene 108: 193-200 (1991)), pBK-CMV, pcDNA3.1 (Invitrogen), and pZeoSV (Stratagene).

Promoters may be cytomegalovirus IE gene promoter and enhancer, SV40 early promoter, a retrovirus LTR such as those from RSV, HIV, and MMLV, and gene promoters from animal cells such as metallothionein, β-actin, elongation factor-1, HSP genes, and such. Alternatively, viral vectors such as those mentioned above may also be used. Viral vectors may be derived from DNA viruses and RNA viruses such as retroviruses, adenoviruses, adeno-associated viruses, herpes viruses, vaccinia viruses, poxviruses, Simbu viruses, Sendai viruses, SV40, and HIV.

Host animal cells may be mouse myeloma cells (e.g. SP2/0, NSO), rat myeloma cells (e.g. YB2/0), mouse hybridoma cells, Namalwa cells (including KJM-1 cells), human embryonic kidney cells (e.g. 293 cells), human leukemia cells (e.g. BALL-1), CHO cells, COS cells (e.g. COS-1, COS-7), hamster embryonic kidney cells (e.g. BHK), mouse Sertoli cells (e.g. TM4), African green monkey kidney cells (e.g. VERO-76), HBT637 cells, HeLa cells, rabbit kidney cells (e.g. MDCK), human liver cells (e.g. HepG2), mouse mammary tumor cells (e.g. MMT060562), TRI cells, MRC cells, FS3 cells, etc.

Methods for introducing expression vectors depend on the type of host cell and vector, but any method can be used as long as it facilitates introduction of antibody-encoding DNA into cells. Vectors can be introduced into prokaryotic cells by methods utilizing calcium ions (Proc. Natl. Acad. Sci. USA 69: 2110 (1972)), protoplasts (Unexamined Published Japanese Patent Application No. (JP-A) Sho 63-24829), electroporation (Gene 17: 107 (1982); Molecular & General Genetics 168: 111 (1979)), and such; introduced into yeast cells by using electroporation (Methods in Enzymology, 194: 182 (1990)), spheroplasts (Proc. Natl. Acad. Sci. USA 81: 4889 (1984)), lithium acetate (J. Bacteriol. 153: 163 (1983)) ), and such; introduced into plant cells by using Agrobacterium (Gene 23: 315 (1983); WO89/05859), sonication (WO91/00358), and such; and into-animal cells by using electroporation (Cytotechnology 3: 133 (1990)), calcium phosphate (JP-A Hei 2-227075), lipofection (Proc. Natl. Acad. Sci. USA 84: 7413 (1987); Virology 52: 456 (1973)), co-precipitation with calcium phosphate, DEAE-dextran, direct injection of DNA using microcapillaries), and such.

Transformants obtained as described above can be cultured, for example, by the following methods:

Culture media for transformants of prokaryotes and eukaryotic microorganisms can be natural or synthetic, as long as the media facilitates efficient culture of the transformants, and comprises utilizable nutrients essential for growth, such as carbon and nitrogen sources, and inorganic salts. Culture may be carried out under aerobic or anaerobic conditions, and other conditions such as temperature, pH of the medium and duration of the culture can be determined appropriately by one skilled in the art, depending on the type of transformant. When using expression vectors equipped with inducible promoters, inducers may be added to the medium as necessary. For example, expression of vectors comprising the lac promoter can be induced by adding IPTG; and expression of vectors comprising the trp promoter can be induced by adding IAA as an inducer.

When using insect cells as hosts, a medium such as the TNM-FH medium (Pharmingen), Sf-900 II SFM (Life Technologies), ExCell400 and ExCell405 (JRH Biosciences), and Grace's Insect Medium (Nature 195: 788 (1962)) can be used. If necessary, antibiotics such as gentamicin may be added to the medium.

For animal cell transformants, common media such as RPMI1640 (The Journal of American Medical Association 199: 519 (1967)), Eagle's MEM (Science 122: 501 (1952)), DMEM (Virology 8: 396 (1959)), and 199 medium (Proceeding of the Society for the Biological Medicine 73: 1 (1950)), or such media added with BSA and the like, can be used. Culture can be carried out under normal conditions such as pH 6 to 8, 30 to 40° C., and 5% CO₂. If necessary, antibiotics such as kanamycin and penicillin may be added to the medium.

The antibodies of the present invention obtained as above can be isolated from within host cells, or from the culture medium if secreted into the extracellular space using signal sequences. They can then be purified as substantially pure polypeptides. The antibodies of the present invention can be separated or purified by appropriately selecting, or combining as necessary, methods generally used in separation and purification. Such methods can be chromatography, filtration, ultrafiltration, salting out, solvent precipitation, solvent extraction, distillation, immunoprecipitation, SDS-polyacrylamide gel electrophoresis, isoelectric point focusing, dialysis, and recrystallization. Chromatography includes affinity chromatography, ion exchange chromatography, hydrophobic chromatography, gel filtration, reverse phase chromatography, absorption chromatography, and the like (Strategies for Protein Purification and Characterization: A Laboratory Course Manual, Daniel R. Marshak et al. eds., Cold Spring Harbor Laboratory Press (1996); Antibodies: A Laboratory Course Manual, Harlow and David Lane eds., Cold Spring Harbor Laboratory Press (1988)). Such chromatographies may be performed using liquid chromatographies such as HPLC, FPLC, and the like. In addition, the antibodies of the present invention may be purified by making use of their affinities towards antigens.

### BRIEF DESCRIPTION OF THE DRAWINGS

FIG. 1 shows photographs of approximately 200 plasma cells or B cells excised from a thin-section frozen specimen. Left: specimen before excision. Right: specimen after excision. The clear white spot in the right photograph shows the portion that was excised.

FIG. 2 shows photographs and graphs representing results of amplifying antibody genes expressed in approximately 200 plasma cells or B cells excised from a thin-section frozen specimen by RT-PCR, and analyzing a portion of the amplification products by electrophoresis using Agilent 2100. The photograph on the left represents the electrophoresis results of (from the left): molecular weight marker, amplification products of the heavy chain variable region (VH6/7-JHmix), and amplification products of the light chain variable region (Vκ2-Jκmix). Graphs on the right show electrophoresis time measured using Agilent 2100. The vertical axis shows the fluorescence intensity and horizontal axis shows the electrophoresis time (second).

FIG. 3 represents results of determining nucleotide sequences for the heavy chain variable regions of antibodies expressed in the approximately 200 plasma cells or B cells excised from a thin-section frozen specimen, and performing a multiple alignment of the amino acid sequences encoded by these nucleotide sequences using ClustalX. The highly conserved positions are shown in the top row of the alignment. Levels of conservation are shown using three symbols, “*”, “:”, and “.”, as follows:
“*” Positions where identical amino acid residues are conserved in all sequences
“:” 1 Positions where an amino acid residue in any one of the following highly conserved groups is present in all sequences
   STA, NEQK, NHQK, NDEQ, QHRK, MILV, MILF, HY, and FYW
“.” Positions where an amino acid residue in any one of the following lowly conserved groups is present in all sequences
   CSA, ATV, SAG; STNK, STPA, SGND, SNDEQK, NDEQHK, NEQHRK, FVLIM, and HFY

FIG. 4 represents results of determining nucleotide sequences for the κ-chain variable regions of antibody genes expressed in the approximately 200 plasma cells or B cells excised from a thin-section frozen specimen, and performing a multiple alignment of the encoded amino acid sequences by ClustalX.

FIG. 5 shows photographs of five plasma cells or B cells excised from a thin-section frozen specimen. Left: specimen before excision. Right: specimen after excision. The white clear portion in the right photograph shows the portion that was excised.

FIG. 6 shows a photograph and a graph representing the results of amplifying antibody genes expressed in the five plasma cells or B cells excised from a thin-section frozen specimen by RT-PCR, and analyzing a portion of the amplification products by electrophoresis using Agilent 2100. Electrophoresis results shown in the left photograph represent results of amplification using the following primer sets (from left to right):
molecular weight marker;
heavy chain variable region (VH6/7-JHmix);
light chain variable region (Vκ1-Jκmix);
light chain variable region (Vκ2-Jκmix);
light chain variable region (Vκ3-Jκmix);
light chain variable region (Vκ4/5-Jκmix); and
light chain variable region (Vκ6/7-Jκmix).

The graph on the right shows electrophoresis time measured using Agilent 2100. The vertical axis shows fluorescence intensity, and the horizontal axis shows electrophoresis time (seconds).

FIG. 7 shows photographs of a single plasma cell excised from a thin-section frozen specimen. Left: specimen before excision. Right: specimen after excision. The white clear portion in the right photograph shows the portion that was excised.

FIG. 8 shows graphs representing results of amplifying antibody genes expressed in a single plasma cell excised from a thin-section frozen specimen by RI-PCR, and analyzing a portion of the amplification products by electrophoresis using Agilent 2100. In the graphs, the vertical axis shows fluorescence intensity, and the horizontal axis shows electrophoresis time (seconds).

FIG. 9 shows photographs of a single plasma cell excised from a thin-section frozen specimen. Left: specimen before excision. Right: specimen after excision. In the left photograph, arrow shows the spot to be excised. The white clear portion in the right photograph shows the portion that was excised.

FIG. 10 shows graphs representing results of amplifying antibody genes expressed in a single plasma cell excised from a thin-section frozen specimen by RI-PCR, and analyzing a portion of the amplification products by electrophoresis using Agilent 2100. In the graphs, the vertical axis shows fluorescence intensity, and the horizontal axis shows electrophoresis time (seconds).

FIG. 11 shows photographs of a single plasma cell excised from a thin-section frozen specimen. Left: specimen before excision. Right: specimen after excision. Arrow in the left photograph shows the spot to be excised. The white clear portion in the right photograph shows the portion that was excised.

FIG. 12 shows a photograph and a graph representing the results of amplifying antibody genes expressed in a single plasma cell excised from a thin-section frozen specimen by RT-PCR, and analyzing a portion of the amplification products by electrophoresis using Agilent 2100. Electrophoresis results in the left photograph represent results of amplification using the following primer sets (from left to right):
molecular weight marker,
heavy chain variable region (VH6/7-JHmix);
light chain variable region (Vκ1-Jκmix);
light chain variable region (Vκ2-Jκmix);
light chain variable region (Vκ3-Jκmix);
light chain variable region (Vκ4/5-Jκmix); and
light chain variable region (Vκ6-Jκmix).

The graph on the right shows electrophoresis time measured using Agilent 2100. The vertical axis shows fluorescence intensity, and the horizontal axis shows electrophoresis time (seconds).

FIG. 13 shows photographs of a single plasma cell excised from a thin-section frozen specimen. Left: specimen before excision. Right: specimen after excision. Arrow in the left photograph shows the spot to be excised. The white clear portion in the right photograph represents the portion that was excised.

FIG. 14 shows graphs representing the results of amplifying antibody genes expressed in the single plasma cell excised from a thin-section frozen specimen by RI-PCR, and analyzing a portion of the amplification products by electrophoresis using Agilent 2100. In the graphs, the vertical axis shows fluorescence intensity, and the horizontal axis shows electrophoresis time (seconds).

FIG. 15 shows photographs of a single plasma cell excised from a thin section of PFA-fixed frozen specimen. Left: specimen before excision. Right: specimen after excision. Cells to be excised are circled in the left photograph. The clear white portion in the right photograph represents the portion that was excised.

FIG. 16 shows graphs representing the results of amplifying antibody genes expressed in a single plasma cell excised from a thin-section PFA-fixed frozen specimen by RT-PCR, and analyzing a portion of the amplification products by electrophoresis using Agilent 2100. In the graphs, the vertical axis shows fluorescence intensity, and the horizontal axis shows electrophoresis time (seconds).

### BEST MODE FOR CARRYING OUT THE INVENTION

Herein below, the present invention will be specifically described using Examples, but it is not to be construed as being limited thereto.

### EXAMPLE 1

### LMD Isolation of a Single Cancer Tissue-Infiltrating B Cell

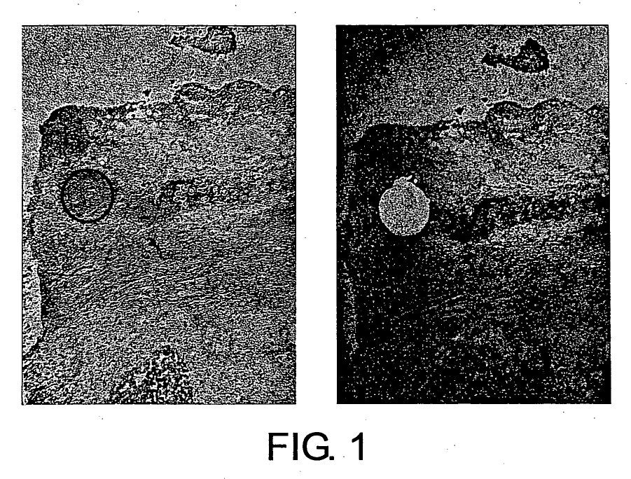

A fresh human tissue (breast cancer tissue) was sliced into a suitable size to prepare frozen blocks using an OCT compound (Tissue-tek), and fixed prior to freezing with fixatives such as paraformaldehyde-lysine-periodate, as necessary. Next, thin specimen sections were prepared from each frozen block and attached onto LMD slides (Matsunami Glass Co.). Resulting thin-section frozen specimens were dried in air, fixed with fixatives such as acetone, and stained with toluidine blue (Muto Pure Chemicals Co., Ltd) or such. After staining, plasma cells were excised with a laser microdissection system (Leica AS-LMD), and recovered using a recovery buffer (RLT solution attached to QIAGEN RNeasy Mini Kit). Plasma cells before and after the excision are shown in FIGS. 1, 5, 7, 9, 11, 13, and 15. In all figures, the left photograph shows the state of the cells before excision, and the right photograph shows the state of the cells after excision. Furthermore, when the cell(s) to be excised can be specified, they are indicated with an arrow or such in the “before excision” photographs (left).

### EXAMPLE 2

### RNA Preparation and cDNA Synthesis

A suspension of approximately one to five B cells excised from a thin-section LMD specimen was mixed with a suspension of about 300 carrier cells which do not express any antibody genes. Total RNAs were prepared from the resultant mixture, using the RNeasy Mini Kit (QIAGEN) according to the manufacturer's instructions. When 50 cells or more were excised from the thin-section specimen, total RNAs were prepared without the addition-of carrier cells. cDNA was synthesized using all 35 μl of the RNA eluate fraction as a template, and Sensiscript Reverse Transcriptase (QIAGEN) according to the manufacturer's instructions. cDNA synthesis reaction was performed on an 80-μl scale using 40 ng of Oligo dT primers (Promega) and 0.8 μg of random hexamers (Invitrogen) as the reverse transcription primers at 37° C. for 1 h. When cDNAs thus synthesized were not immediately subjected to PCR, they were stored at −80° C.

### EXAMPLE 3

### Cloning of Human Antibody Variable Regions

PCR primers to be used for cloning human antibody variable regions were designed based on the research paper “J. Mol. Biol. (1991) 222, 581-597”, and the web site of Medical Research Council (MRC), “V BASE” (http://www.mrc-cpe.cam.ac.uk/vbase-ok.php?menu=901). These nucleotide sequences are shown below (SEQ ID NOs: 97 to 150). Primers for cloning heavy chain variable regions are designated with VH and JH as the initial letters. Primers for amplifying the light chain κ-chain or the light chain λ-chain are designated with VK and JK, or VL and JL as the initial letters.

| | |
|---|---|
| VH1a | 5′-CAGGT(GT)CAGCTGGTGCAGTCTGG-3′ |
| VH1b | 5′-CAGGTCCAGCTTGTGCAGTCTGG-3′ |
| VH1c | 5′-(GC)AGGTCCAGCTGGTACAGTCTGG-3′ |
| VH1d | 5′-CA(AG)ATGCAGCTGGTGCAGTCTGG-3′ |
| VH2a | 5′-CAGATCACCTTGAAGGAGTCTGGT-3′ |
| VH2b | 5′-CAGGTCACCTTGA(AG)GGAGTCTGGT-3′ |
| VH3a | 5′-GA(AG)GTGCAGCTGGTGGAGTCTGG-3′ |
| VH3b | 5′-CAGGTGCAGCTGGTGGAGTCTGG-3′ |
| VH3c | 5′-GAGGTGCAGCTGTTGGAGTCTGG-3′ |
| VH4a | 5′-CAG(CG)TGCAGCTGCAGGAGTCGGGC-3′ |
| VH4b | 5′-CAGGTGCAGCTACAGCAGTGGGGC-3′ |
| VH5a | 5′-GA(AG)GTGCAGCTGGTGCAGTCTGGA-3′ |
| VH6a | 5′-CAGGTACAGCTGCAGCAGTCAGGT-3′ |
| VH7a | 5′-CAGGT(CG)CAGCTGGTGCAATCTGG-3′ |
| JH1245 | 5′-TGAGGAGACGGTGACCAGGGT(GT)CC-3′ |
| JH3 | 5′-TGAAGAGACGGTGACCATTGTCCC-3′ |
| JH6 | 5′-TGAGGAGACGGTGACCGTGGTCCC-3′ |
| VK1a | 5′-(AG)ACATCCAGATGACCCAGTCTCCA-3′ |
| VK1b | 5′-G(AC)CATCCAGTTGACCCAGTCTCCA-3′ |
| VK1c | 5′-GCCATCC(AG)GATGACCCAGTCTCCA-3′ |
| VK1d | 5′-GTCATCTGGATGACCCAGTCTCCA-3′ |
| VK2a | 5′-GATATTGTGATGACCCAGACTCCA-3′ |
| VK2b | 5′-GAT(AG)TTGTGATGACTCAGTCTCCA-3′ |
| VK3a | 5′-GAAATTGTGTTGAC(AG)CAGTCTCCA-3′ |
| VK3b | 5′-GAAATAGTGATGACGCAGTCTCCA-3′ |
| VK3c | 5′-GAAATTGTAATGACACAGTCTCCA-3′ |
| VK4a | 5′-GACATCGTGATGACCCAGTCTCCA-3′ |
| VK5a | 5′-GAAACGACACTCACGCAGTCTCCA-3 |
| VK6a | 5′-GAAATTGTGCTGACTCAGTCTCCA-3′ |
| VK6b | 5′-GATGTTGTGATGACACAGTCTCCA-3′ |
| JK1 | 5′-ACGTTTGATTTCCACCTTGGTCCC-3′ |
| JK24 | 5′-ACGTTTGATCTCCA(CG)CTTGGTCCC-3′ |
| JK3 | 5′-ACGTTTGATATCCACTTTGGTCCC-3′ |
| JK5 | 5′-ACGTTTAATCTCCAGTCGTGTCCC-3′ |
| VL1a | 5′-CAGTCTGTGCTGAGTCAGCCACCC-3′ |
| VL1b | 5′-CAGTCTGTG(GT)TGACGCAGCCGCCC-3′ |
| VL2 | 5′-CAGTCTGCCCTGACTCAGCCT(CG)-3′ |
| VL3a | 5′-TCCTATG(AT)GCTGACTCAGCCACCC-3′ |
| VL3b | 5′-TCCTATGAGCTGACACAGC(CT)ACCC-3′ |
| VL3c | 5′-TCTTCTGAGCTGACTCAGGACCCT-3′ |
| VL3d | 5′-TCCTATGAGCTGATGCAGCCACCC-3′ |
| VL4a | 5′-CAGCCTGTGCTGACTCAATCATCC-3′ |
| VL4b | 5′-CAGCTTGTGCTGACTCAATCGCCC-3′ |
| VL4c | 5′-CTGCCTGTGCTGACTCAGCCCCCG-3′ |
| VL5a | 5′-CAGCCTGTGCTGACTCAGCCA(CT)CT-3′ |
| VL5c | 5′-CAGGCTGTGCTGACTCAGCCGGCT-3′ |
| VL6 | 5′-AATTTTATGCTGACTCAGCCCCAC-3′ |
| VL7 | 5′-CAG(AG)CTGTGGTGACTCAGGAGCCC-3′ |
| VL8 | 5′-CAGACTGTGGTGACCCAGGAGCCA-3′ |
| VL4_9 | 5′-C(AT)GCCTGTGCTGACTCAGCCACCT-3′ |
| VL10 | 5′-CAGGCAGGGCTGACTCAGCCACCC-3′ |
| JL1 | 5′-ACCTAGGACGGTGACCTTGGTCCC-3′ |
| JL23 | 5′-ACCTAGGACGGTCAGCTTGGTCCC-3′ |
| JL7 | 5′-ACCGAGGACGGTCAGCTGGGTGCC-3′ |

For cloning the heavy chain variable regions, _78 -chain variable regions, and λ-chain variable regions, PCR amplifications were carried out using combinations of the Taq DNA polymerase Core Kit (QIAGEN) and primer mixtures for each gene subset. For amplifying the heavy chain and κ-chain, five different primer mixtures were prepared for each. Ten kinds of reaction solutions were prepared using these primer mixtures. Combinations of primers in the mixtures are shown in Table 1. Prepared reaction mixtures (20 μl) had a final concentration of 1× reaction buffer containing 4 [2l of the template cDNA, 1× Q solution (QIAGEN), 0.4 M dNTP, 0.4 μM each of the forward and reverse primers, and 2 U Taq DNA polymerase. The reaction mixture was subjected to 40 cycles of amplification reaction on Applied Biosystems PE9700. The amplification cycle was carried out under conditions of denaturation at 94° C. for 10 sec, annealing at 50° C. for 30 sec, and elongation at 72° C. for 30 sec.

TABLE 1

| PRIMER SET | FORWARD | | | | REVERSE | | | |
|---|---|---|---|---|---|---|---|---|
| VH1-JH MIX | VH1a | VH1b | VH1c | VH1d | JH1245 | JH3 | JH6 | |
| VH2-JH MIX | VH2a | VH2b | | | JH1245 | JH3 | JH6 | |
| VH3/5-JH MIX | VH3a | VH3b | VH3c | VH3d | JH1245 | JH3 | JH6 | |
| VH4-JH MIX | VH4a | VH4b | | | JH1245 | JH3 | JH6 | |
| VH6/7-JH MIX | VH6a | VH7a | | | JH1245 | JH3 | JH6 | |
| VK1-JK MIX | VK1a | VK1b | VK1c | VK1d | JK1 | JK24 | JK3 | JK5 |
| VK2-JK MIX | VK2a | VK2b | | | JK1 | JK24 | JK3 | JK5 |
| VK3-JK MIX | VK3a | VK3b | VK3c | | JK1 | JK24 | JK3 | JK5 |
| VK4/5-JK MIX | VK4a | VK5a | | | JK1 | JK24 | JK3 | JK5 |
| VK6-JK MIX | VK6a | VK6b | | | JK1 | JK24 | JK3 | JK5 |

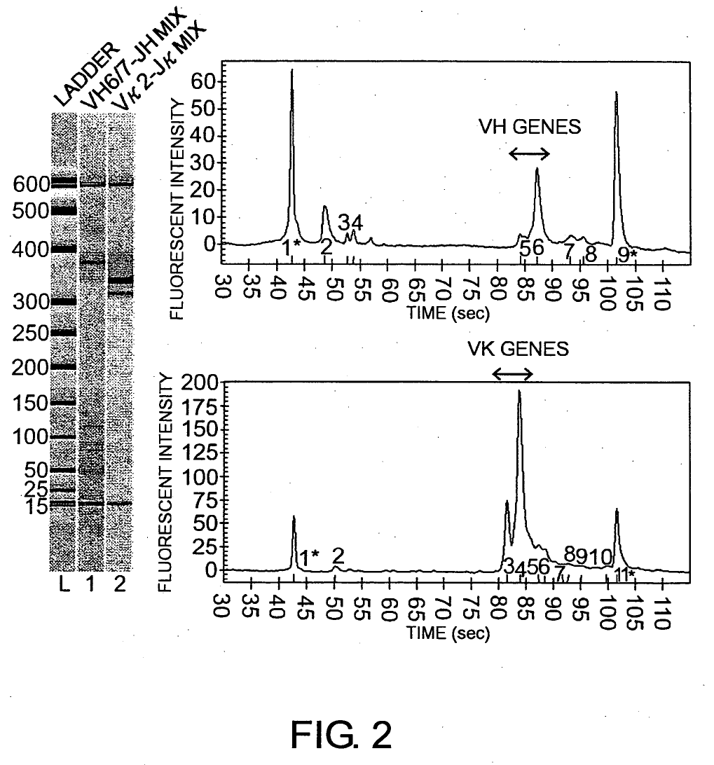

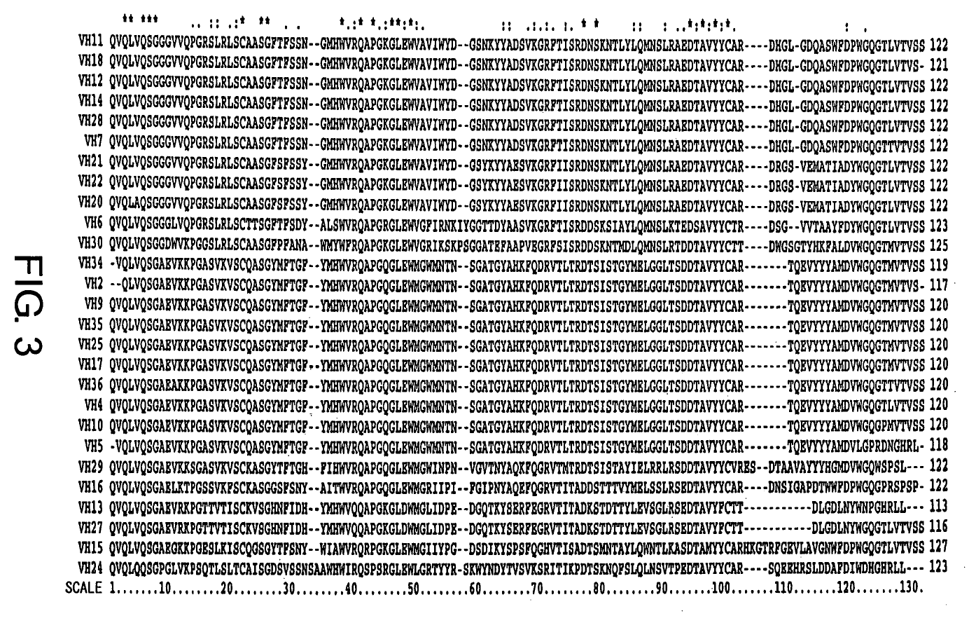

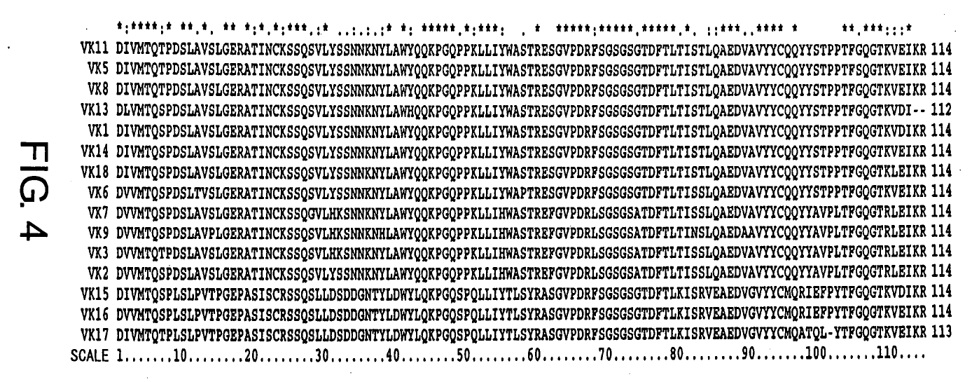

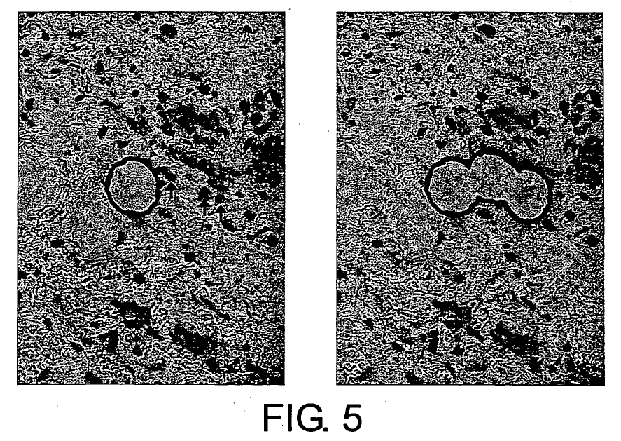

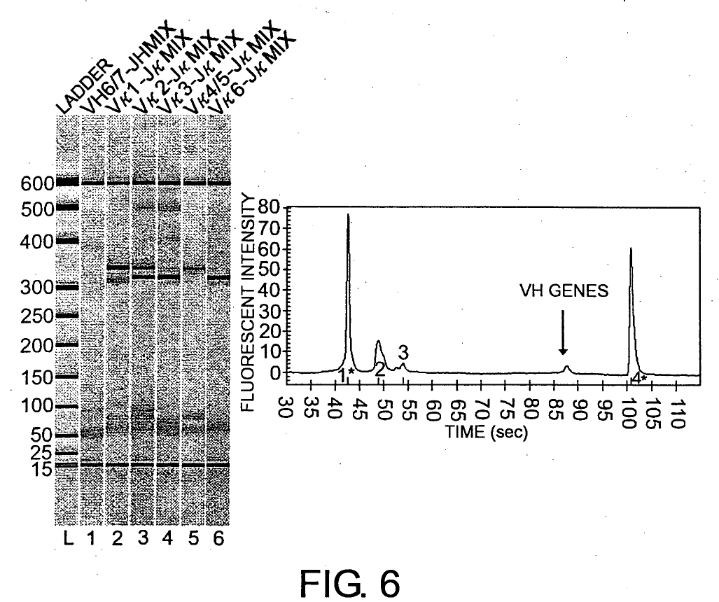

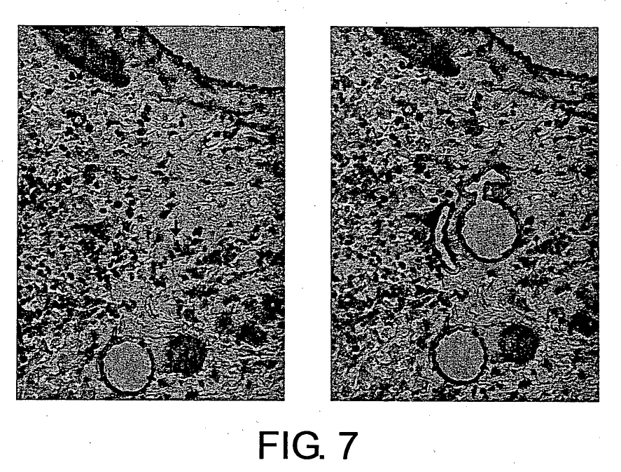

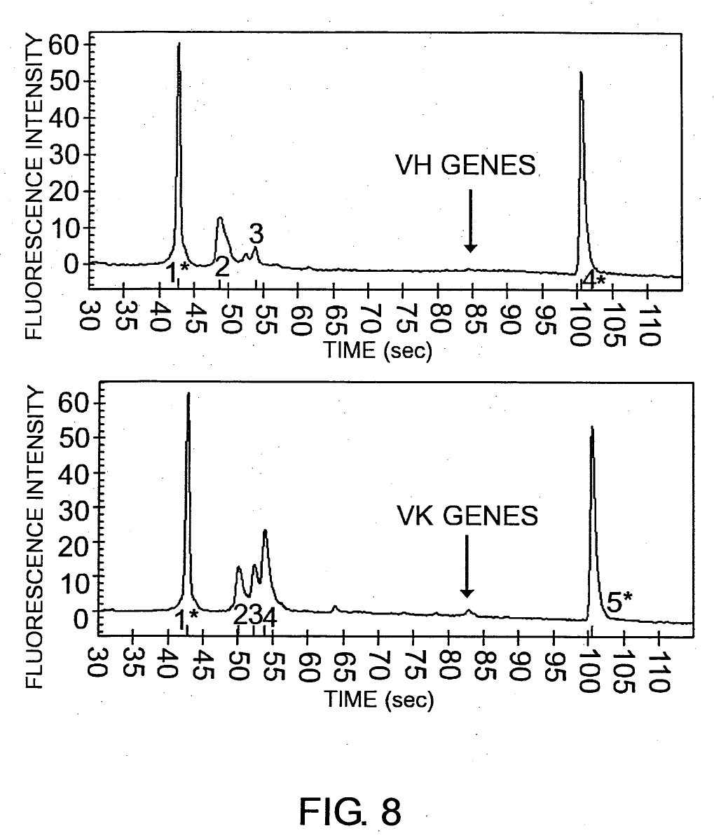

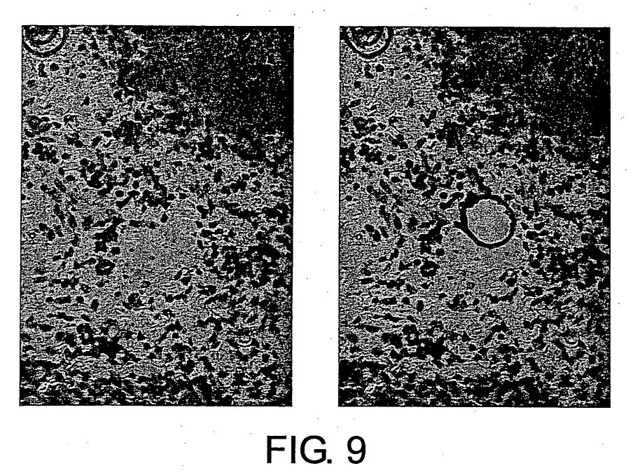

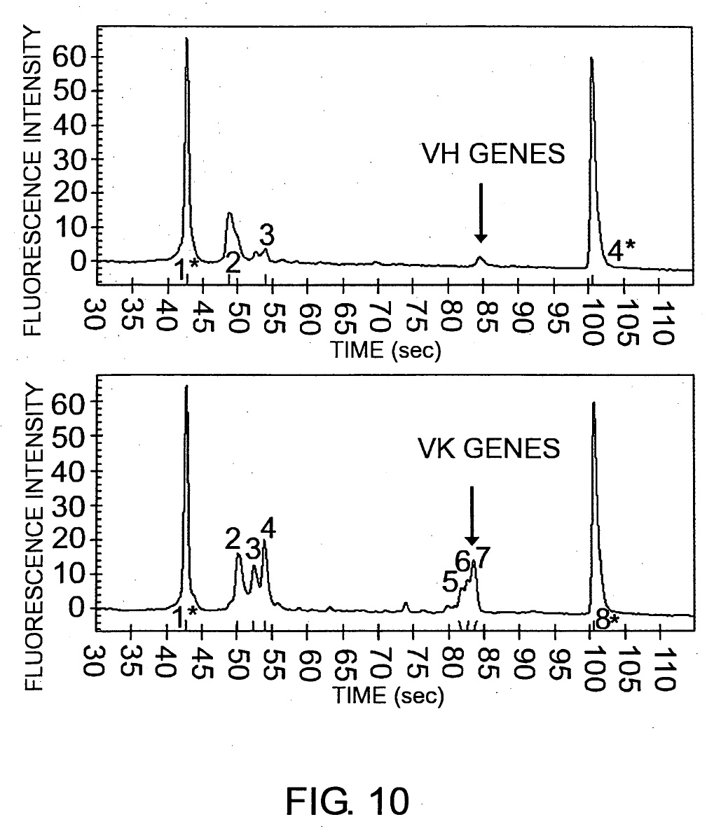

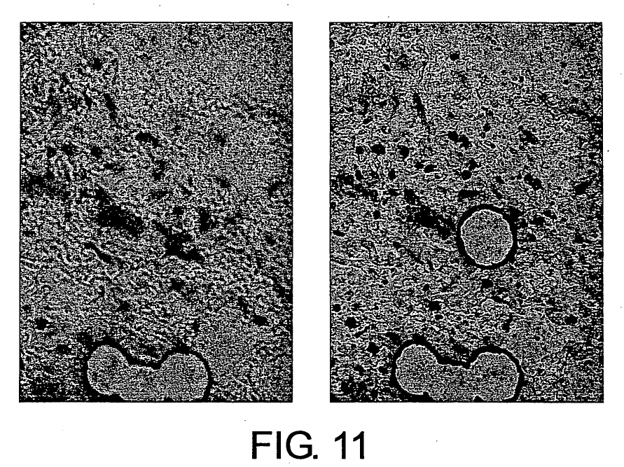

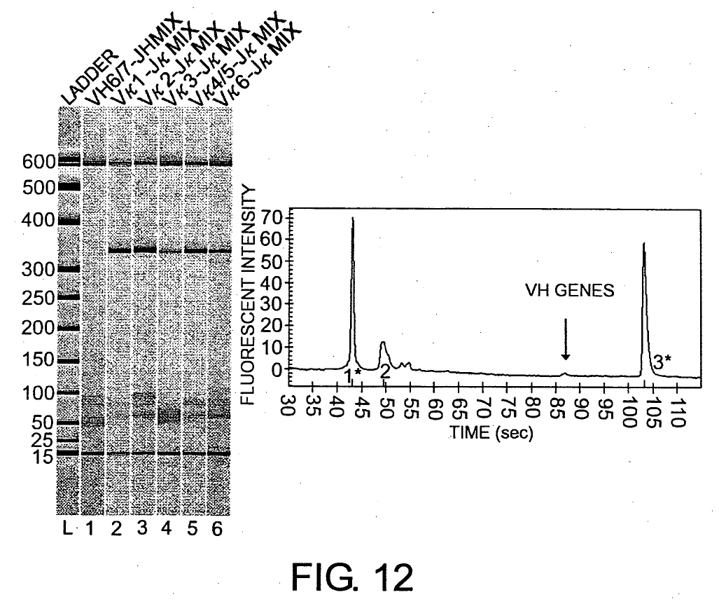

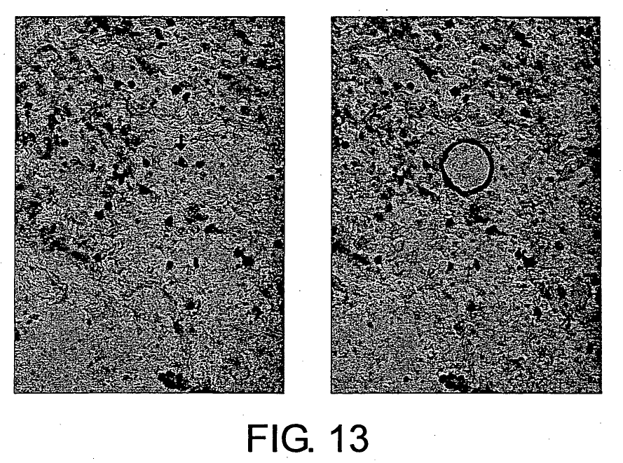

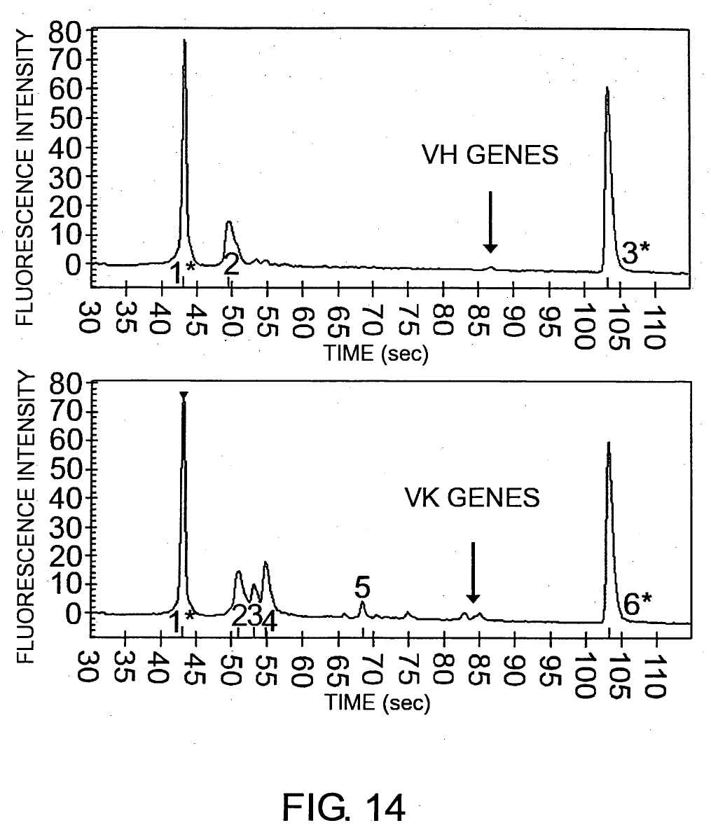

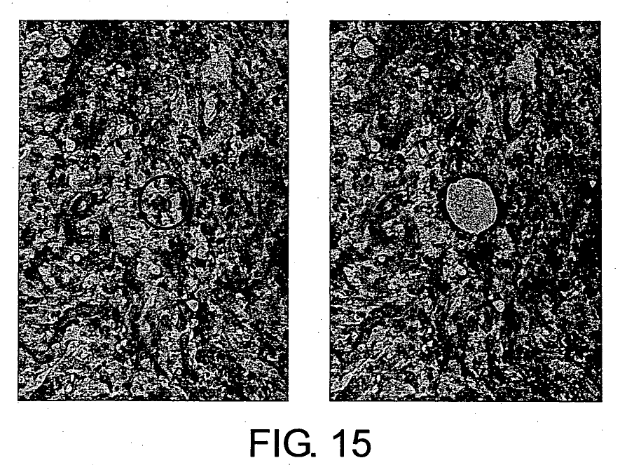

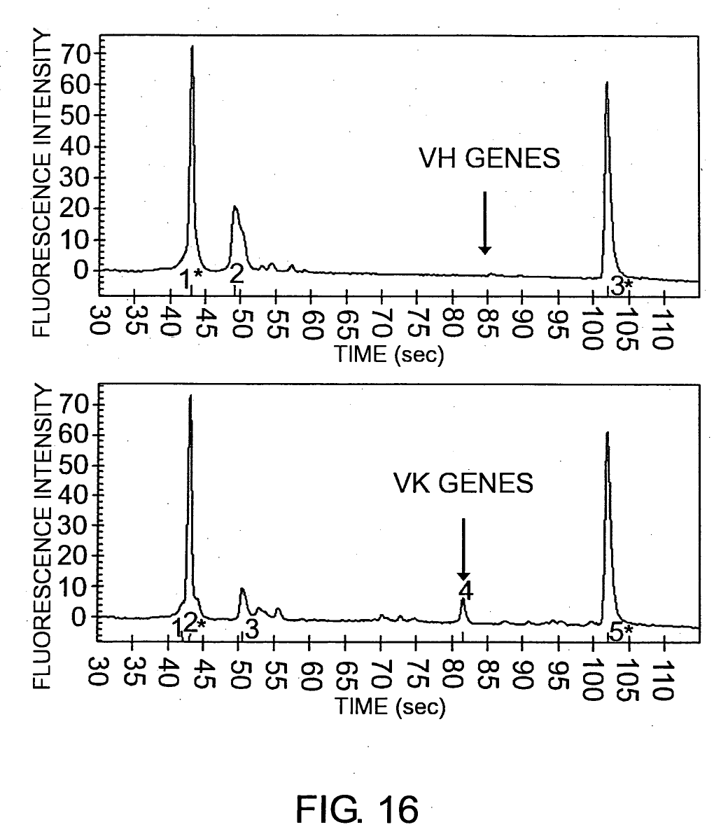

Following the reaction, amplification products were analyzed using DNA 7500 Labchip and Agilent 2100. Results of the amplification were shown in FIGS. 2, 6, 8, 10, 12, 14, and 16. Amplification products were purified using the QIAGEN PCR Purification Kit. When the amount of PCR-amplified products was small, the products were electrophoresed on an agarose gel, and the region corresponding to the molecular weight of the antibody variable region gene was excised and then amplified again. DNA fragments thus obtained were cloned into pGEM-T Easy (Promega), and then introduced into E. coli DH5α. Nucleotide sequences of the insert sequences in the recombinant plasmids were determined and it was confirmed that antibody genes were amplified. The nucleotide sequences thus determined are shown in SEQ ID NOs: 1 to 54 (heavy chains) and SEQ ID NOs: 55 to 84 (light chains). Furthermore, the determined amino acid sequences were aligned as shown in FIGS. 3 and 4. The alignment results confirmed that multiple clones of the antibody genes were obtained by the methods of this invention. However, since the isolated variable regions do not have diverse types of amino acid sequences, B cells present in the specimens may very likely have been cell populations that had multiplied on specific antigen stimulations.

### EXAMPLE 4

### Preparation of Single-Stranded Antibody Molecules

The linker sequences to be used for preparing single-stranded antibody genes were produced according to the method of Marks et al. (J. Mol. Biol. (1991) 222, 581-597). The template DNA sequences and the nucleotide sequences of primers used in the preparation are shown below. Linker fragments synthesized using PCR amplification were confirmed by agarose gel electrophoresis, and bands containing the respective fragments were excised and purified.

| Template DNA sequence (Template linker)/SEQ ID NO: 151 | |
|---|---|
| 5′-GGACAATGGTCACCGTCTCTTCAGGTGGTGGTGGTTCGGGTGGTGGTGGTTCGGGTG | |
| GTGGCGGATCGGACATCCAGATGACCCAGTCTCC-3′ | |
| Nucleotide sequences of primers: | |
| Reverse JH for linker/SEQ ID NO: 152 to 155 | |
| 1 LJH1_2 | 5′-GCACCCTGGTCACCGTCTCCTCAGGTGG-3′ |
| 2 LJH3 | 5′-GGACAATGGTCACCGTCTCTTCAGGTGG-3′ |
| 3 LJH4_5 | 5′-GAACCCTGGTCACCGTCTCCTCAGGTGG-3′ |
| 4 LJH6 | 5′-GGACCACGGTCACCGTCTCCTCAGGTGG-3′ |
| Reverse VK for linker/SEQ ID NO: 156 to 161 | |
| 5 LVK1 | 5′-GGAGACTGGGTCATCTGGATGTCCGATCCGCC-3′ |
| 6 LVK2 | 5′-GGAGACTGAGTCATCACAACATCCGATCCGCC-3′ |
| 7 LVK3 | 5′-GGAGACTGCGTCAACACAATTTCCGATCCGCC-3′ |
| 8 LVK4 | 5′-GGAGACTGGGTCATCACGATGTCCGATCCGCC-3′ |
| 9 LVK5 | 5′-GGAGACTGCGTGAGTGTCGTTTCCGATCCGCC-3′ |
| 10 LVK6 | 5′-GGAGACTGAGTCAGCACAATTTCCGATCCGCC-3′ |
| Reverse VL for linker/SEQ ID NO: 162 to 168 | |
| 11 LVL1 | 5′-GGCGGCTGCGTCAACACAGACTGCGATCCGCCACCGCCAGAG-3′ |
| 12 LVL2 | 5′-GCAGGCTGAGTCAGAGCAGACTGCGATCCGCCACCGCCAGAG-3′ |
| 13 LVL3a | 5′-GGTGGCTGAGTCAGCACATAGGACGATCCGCCACCGCCAGAG-3′ |
| 14 LVL3b | 5′-GGGTCCTGAGTCAGCTCAGAAGACGATCCGCCACCGCCAGAG-3′ |
| 15 LVL4 | 5′-GGCGGTTGAGTCAGTATAACGTGCGATCCGCCACCGCCAGAG-3′ |
| 16 LVL5 | 5′-GACGGCTGAGTCAGCACAGACTGCGATCCGCCACCGCCAGAG-3′ |
| 17 LVL6 | 5′-TGGGGCTGAGTCAGCATAAAATTCGATCCGCCACCGCCAGAG-3′ |

PCR using the primer sets (SEQ ID NOs: 169 to 182) as shown below was performed by mixing the heavy chain variable region, κ-chain variable region or λ-chain variable region, and linker sequence, which were PCR-amplified using the cDNA synthesized from mRNA extracts of a single B cell as a template. For κ-chain light chains, reaction solutions were prepared using a combination of VH and JK primers. For λ-chain light chains, the reaction solutions were prepared with a combination of VH and JL primers. These reaction mixtures were prepared using KOD plus DNA polymerase (TOYOBO) according to manufacturer's instructions. Prior to the addition of primers, seven cycles of denaturation (at 94° C. for 15 sec) and elongation (at 68° C. for 1 min) were performed, and after that, 20 cycles of denaturation (at 94° C. for 15 sec) and elongation (at 68° C. for 1 min) followed.

| | |
|---|---|
| VH1BACKNco | 5′-AGTATTGACCATGGCCCAGGTGCAGCTGGTGCAGTCTGG-3′ |
| VH2BACKNco | 5′-AGTATTGACCATGGCCCAGGTCAACTTAAGGGAGTCTGG-3′ |
| VH3BACKNco | 5′-AGTATTGACCATGGCCGAGGTGCAGCTGGTGGAGTCTGG-3′ |
| VH4BACKNco | 5′-AGTATTGACCATGGCCCAGGTGCAGCTGCAGGAGTCGGG-3′ |
| VH5BACKNco | 5′-AGTATTGACCATGGCCCAGGTGCAGCTGTTGCAGTCTGC-3′ |
| VH6BACKNco | 5′-AGTATTGACCATGGCCCAGGTACAGCTGCAGCAGTCAGG-3′ |
| JK1FOREco | 5′-TAATGAATTCACGTTTGATTTCCACCTTGGTCCC-3′ |
| JK2FOREco | 5′-TAATGAATTCACGTTTGATCTCCAGCTTGGTCCC-3′ |
| Jk3FOREco | 5′-TAATGAATTCACGTTTGATATCCACTTTGGTCCC-3′ |
| JK4FOREco | 5′-TAATGAATTCACGTTTGATCTCCACCTTGGTCCC-3′ |
| JK5FOREco | 5′-TAATGAATTCACGTTTAATCTCCAGTCGTGTCCC-3′ |
| JL1FOREco | 5′-TAATGAATTCACCTAGGACGGTGACCTTGGTCCC-3′ |
| JL2_3FOREco | 5′-TAATGAATTCACCTAGGACGGTCAGCTTGGTCCC-3′ |
| JL4_5FOREco | 5′-TAATGAATTCACCTAAAACGGTGAGCTGGGTCCC-3′ |

After the presence of the amplification products were confirmed by agarose gel electrophoresis, bands containing the corresponding gene fragments were excised and purified. Gene fragments thus excised were cleaved with restriction enzymes and inserted into expression vectors. The expression vectors were designed such that the inserts can be expressed under the control of a T7 promoter and a FLAG tag can be added to the C terminus of the recombinants. The expression vectors thus obtained were introduced into E. coli DH5α. The expression plasmids were confirmed to have the inserts by DNA sequencing, and then introduced into the E. coli BL21 (DE3) strain.

The nucleotide sequences of the single-stranded antibodies constructed in this Example, and their translated amino acid sequences, are shown in SEQ ID NOs: 183 to 188. 70-6scFV (SEQ ID NOs: 187-188) is a single-stranded antibody prepared from a heavy chain (SEQ ID NOs: 93-94) and a light chain (SEQ ID NOs: 95-96) isolated from a single B cell. On the other hand, 70-5AscFv (SEQ ID NOs: 183-184) and 70-5BscFv (SEQ ID NOs: 185-186) are single-stranded antibodies formed by combining heavy chains and light chains obtained from five B cells. The nucleotide sequences and amino acid sequences that constitute the heavy chains comprised in 70-5AscFv and 70-5BscFv are shown in SEQ ID NOs: 91-92, and the nucleotide sequences and amino acid sequences which constitute the light chains are shown in SEQ ID NOs: 85-88.

Recombinant single-stranded antibodies were prepared from E. coli culture supernatants. During the logarithmic growth phase of transformed E. coli cells, expression of the recombinant antibodies was induced by the addition of 0.5 mM isopropyl-β-thiogalactopyranoside at 30° C. After overnight culture, the culture solution was centrifuged to separate the culture supernatant from cells. Following filtration, the resulting supernatant was applied to Anti-FLAG M2 affinity column (Sigma) and the recombinant protein was absorbed thereto. After washing the column, the recombinant protein was eluted with 0.1 M glycine (pH3.5). The eluate was applied to PD10 column (Amersham Pharmacia Biotech), and the buffer was immediately replaced with PBS containing 0.01% Tween-20. Presence of the protein was confirmed by Coomassie staining or Western blotting with anti-FLAG antibodies following SDS-PAGE.

### EXAMPLE 3

### Immunostaining

Flash-frozen sections of a cancer tissue were fixed with PBS solution containing 1% paraformaldehyde for 10 min. The endogenous peroxidase activity was blocked with 0.3% hydrogen peroxide. In order to avoid non-specific bindings of recombinant antibodies, tissue sections were blocked with 10% fetal bovine serum prior to incubation with a solution containing recombinant antibodies. The solution containing recombinant antibodies was diluted in PBS containing 1% BSA and 0.1% Tween-20, and tissue sections were incubated in this solution. The bound recombinant antibodies were detected by the conversion of 3,3-diamino-benzidine-teetrahydrochloride with the peroxidase-conjugated anti-FLAG antibody (FLAG) into a brown precipitate in the presence of hydrogen peroxide. Before and after the addition of antibodies, washing was carried out three times with PBS containing 0.1% Tween-20 at room temperature for 5 min. Prior to mounting, tissue sections were counterstained with hematoxylin, and dehydrated using ethanol and xylene. As a negative control for antibody staining, similar procedures were performed without adding the recombinant antibodies.

### INDUSTRIAL APPLICABILITY

The present invention enables the isolation of polynucleotides encoding antibodies against a lesion without depending on B cell cloning. Since the methods of this invention do not rely on B cell cloning, they enable easy acquisition of genes derived from human antibody-producing cells which are difficult to clone.

Based on the present invention, genes encoding cancer tissue-recognizing antibodies can be isolated from B cells that infiltrate the cancer tissue. Antibodies recognizing cancer cells are useful for the diagnosis and treatment of cancer. By using this invention, antibody genes can be easily obtained from human antibody-producing cells as well. Acquisition of genes for cancer tissue-recognizing human antibodies is of great significance in cancer diagnosis and treatment.

For diagnosis and treatment of cancer using antibodies, antibodies are administered to humans. For example, to diagnose cancer using antibodies, antibody molecules with a traceable label are administered so as to indicate the presence of cancer at the region where the antibodies are localized. For cancer treatment, antibodies are utilized in target therapy. That is, antibodies conjugated with an anticancer agent are administered to patients. Human antibodies can be expected to be highly safe when administered to humans. In addition, since human antibodies are unlikely to be recognized as foreign proteins, the concentration can be stably maintained in blood over a long period of time.

All prior art documents cited herein are incorporated by reference into the present description.

1. A method for isolating a polynucleotide encoding an antibody against a lesional tissue, wherein the method comprises the steps of:
(a) isolating a lesional tissue-infiltrating B cell; and
(b) obtaining a polynucleotide encoding an antibody from the isolated B cell.

2. The method of claim 1, wherein the lesional tissue is a cancer tissue.

3. The method of claim 1, wherein step (a) of isolating a lesional tissue-infiltrating B cell comprises the step of excising a region comprising a B cell from a section of said lesional tissue.

4. The method of claim 1, wherein step (b) of obtaining a polynucleotide encoding an antibody comprises the step of amplifying a gene encoding an antibody variable region.

5. An antibody-encoding polynucleotide isolated by the method of claim 1.

6. The polynucleotide of claim 5, wherein the antibody-encoding polynucleotide comprises a polynucleotide encoding an antibody variable region.

7. An expression vector comprising the polynucleotide of claim 5.

8. A host cell comprising the polynucleotide of claim 5.

9. A method for producing an antibody, wherein the method comprises the steps of:
culturing the host cell of claim 8; and
recovering an antibody which is the expression product.

10. An antibody produced by the method of claim 9.

11. An antibody encoded by the polynucleotide of claim 5.

12. The antibody production method of claim 9, wherein the method further comprises the steps of:
(1) contacting the antibody obtained by the method of claim 9 with a lesional tissue;
(2) detecting the binding between the antibody and the lesional tissue; and
(3) selecting an antibody that binds to the lesional tissue.

13. A host cell comprising the vector of claim 7.


SEQUENCE LISTING (188 sequences)

SEQ ID NO: 1 - DNA, 360 bp, Homo sapiens
  CDS (1)..(360)
caggtgcagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggccaatggtcaccgtctcttca

SEQ ID NO: 2 - protein, 120 aa, Homo sapiens
QVQLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGPMVTVSS

SEQ ID NO: 3 - DNA, 366 bp, Homo sapiens
  CDS (1)..(366)
caggtccagctggtgcaatctggaggaggcgtggtccagcctgggaggtccctgagactctcctgtgcagcctctggattcaccttcagtagcaatggcatgcactgggtccgccaggctccaggcaaggggctggagtgggtggcagttatatggtatgatggaagtaataaatactatgcagactccgtgaagggccgattcaccatctccagagacaattccaagaacacactgtatctgcaaatgaacagcctgagagccgaggacacggctgtgtattactgtgcgagagatcacggccttggtgatcaagcctcctggttcgacccctggggccagggaaccctggtcaccgtctcctca

SEQ ID NO: 4 - protein, 122 aa, Homo sapiens
QVQLVQSGGGVVQPGRSLRLSCAASGFTFSSNGMHWVRQAPGKGLEWVAVIWYDGSNKYYADSVKGRFTISRDNSKNTLYLQMNSLRAEDTAVYYCARDHGLGDQASWFDPWGQGTLVTVSS

SEQ ID NO: 5 - DNA, 366 bp, Homo sapiens
  CDS (1)..(366)
caggtccagctggtgcaatctgggggaggcgtggtccagcctgggaggtccctgagactctcctgtgcagcctctggattcaccttcagtagcaatggcatgcactgggtccgccaggctccaggcaaggggctggagtgggtggcagttatatggtatgatggaagtaataaatactatgcagactccgtgaagggccgattcaccatctccagagacaattccaagaacacactgtatctgcaaatgaacagcctgagagccgaggacacggctgtgtattactgtgcgagagatcacggccttggtgatcaagcctcctggttcgacccctggggccagggaaccctggtcaccgtctcctca

SEQ ID NO: 6 - protein, 122 aa, Homo sapiens; identical to SEQ ID NO: 4

SEQ ID NO: 7 - DNA, 340 bp, Homo sapiens
  CDS (1)..(339)
caggtgcagctggtgcaatctggggctgaggtgaggaagcctgggacgacagtgacaatctcctgcaaggtttctggacacaacttcatcgaccactacatgcattgggtacaacaggcccctggaaaagggcttgactggatgggactaattgaccctgaagatggtcagacgaaatattcagagaggtttgagggcagagtcacaattaccgcggacaagtcaacagacacaacctacttggaggtgagcggcctgagatcggaagacacggccgtttatttctgtacaacggacttgggtgacttgaattattggaaccctggtcaccgtctcctca

SEQ ID NO: 8 - protein, 113 aa, Homo sapiens
QVQLVQSGAEVRKPGTTVTISCKVSGHNFIDHYMHWVQQAPGKGLDWMGLIDPEDGQTKYSERFEGRVTITADKSTDTTYLEVSGLRSEDTAVYFCTTDLGDLNYWNPGHRLL

SEQ ID NO: 9 - DNA, 366 bp, Homo sapiens; identical to SEQ ID NO: 5
  CDS (1)..(366)

SEQ ID NO: 10 - protein, 122 aa, Homo sapiens; identical to SEQ ID NO: 4

SEQ ID NO: 11 - DNA, 381 bp, Homo sapiens
  CDS (1)..(381)
caggtccagctggtgcaatctggagctgaggggaaaaagccgggagagtctctgaagatctcctgtcagggttctggatacacatttagcaattactggatcgcctgggtgcgccagaggcccgggaaaggcctggagtggatggggatcatctatcctggtgactctgatatcaaatacagtccgtccttccaaggccatgtcaccatctcagccgacacgtccatgaacaccgcctacctgcagtggaacaccctgaaggcctcggacaccgccatgtactactgtgcgagacataaagggaccaggttcggggaggttttggcggttggcaactggttcgacccctggggccagggaaccctggtcaccgtctcctca

SEQ ID NO: 12 - protein, 127 aa, Homo sapiens
QVQLVQSGAEGKKPGESLKISCQGSGYTFSNYWIAWVRQRPGKGLEWMGIIYPGDSDIKYSPSFQGHVTISADTSMNTAYLQWNTLKASDTAMYYCARHKGTRFGEVLAVGNWFDPWGQGTLVTVSS

SEQ ID NO: 13 - DNA, 368 bp, Homo sapiens
  CDS (1)..(366)
caggtccagctggtgcaatctggggctgagttgaagacgcctgggtcctcggtgaaattctcctgcaaggcttccggaggcagcttcagcaactatgctatcacctgggtgcgacaggcccctggacaaggtcttgagtggatgggaaggatcatccctatctttggtataccaaactacgcacaggaattccagggcagagtcacgattaccgccgacgattccacgaccacagtctacatggaactgagcagcctgagatctgaggacacggccgtgtattactgtgcgagagataattcaataggagcacctgatacttggtggttcgacccctggggccagggaccacggtcaccgtctcctca

SEQ ID NO: 14 - protein, 122 aa, Homo sapiens
QVQLVQSGAELKTPGSSVKFSCKASGGSFSNYAITWVRQAPGQGLEWMGRIIPIFGIPNYAQEFQGRVTITADDSTTTVYMELSSLRSEDTAVYYCARDNSIGAPDTWWFDPWGQGPRSPSP

SEQ ID NO: 15 - DNA, 360 bp, Homo sapiens
  CDS (1)..(360)
caggtccagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggacaatggtcaccgtctcttca

SEQ ID NO: 16 - protein, 120 aa, Homo sapiens
QVQLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGTMVTVSS

SEQ ID NO: 17 - DNA, 365 bp, Homo sapiens
  CDS (1)..(363)
caggtccagctggtgcaatctgggggaggcgtggtccagcctgggaggtccctgagactctcctgtgcagcctctggattcaccttcagtagcaatggcatgcactgggtccgccaggctccaggcaaggggctggagtgggtggcagttatatggtatgatggaagtaataaatactatgcagactccgtgaagggccgattcaccatctccagagacaattccaagaacacactgtatctgcaaatgaacagcctgagggccgaggacacggctgtgtattactgtgcgagagatcacggccttggtgatcaagcctcctggttcgacccctggggccagggaaccctggtcaccgtctcctc

SEQ ID NO: 18 - protein, 121 aa, Homo sapiens
QVQLVQSGGGVVQPGRSLRLSCAASGFTFSSNGMHWVRQAPGKGLEWVAVIWYDGSNKYYADSVKGRFTISRDNSKNTLYLQMNSLRAEDTAVYYCARDHGLGDQASWFDPWGQGTLVTVS

SEQ ID NO: 19 - DNA, 366 bp, Homo sapiens
  CDS (1)..(366)
caggtccagctggcgcaatctggaggaggcgtggtccagcctgggaggtccctgagactctcctgtgcagcctctggattcagcttcagtagctatggcatgcactgggtccgccaggctccaggcaaggggctggagtgggtggcagttatatggtatgatggaagctataaatactatgcagaatccgtgaagggccgattcatcatctccagagacaattccaagaacaccctgtatctgcaaatgaacagcctgagagccgaggacacggctgtctattactgtgcgagagatcgggggtcggtggagatggctacaatcgcggactactggggccagggaaccctggtcaccgtctcctca

SEQ ID NO: 20 - protein, 122 aa, Homo sapiens
QVQLAQSGGGVVQPGRSLRLSCAASGFSFSSYGMHWVRQAPGKGLEWVAVIWYDGSYKYYAESVKGRFIISRDNSKNTLYLQMNSLRAEDTAVYYCARDRGSVEMATIADYWGQGTLVTVSS

SEQ ID NO: 21 - DNA, 366 bp, Homo sapiens
  CDS (1)..(366)
caggtccagctggtgcaatctgggggaggcgtggtccagcctgggaggtccctgagactctcctgtgcagcctctggattcagcttcagtagctatggcatgcactgggtccgccaggctccaggcaaggggctggagtgggtggcagttatatggtatgatggaagttataaatactatgcagaatccgtgaagggccgattcatcatctccagagacaattccaagaacaccctgtatctgcaaatgaacagcctgagagccgaggacacggctgtgtattactgtgcgagagatcgggggtcggtagagatggctacaatcgcggactactggggccagggaaccctggtcaccgtctcctca

SEQ ID NO: 22 - protein, 122 aa, Homo sapiens
QVQLVQSGGGVVQPGRSLRLSCAASGFSFSSYGMHWVRQAPGKGLEWVAVIWYDGSYKYYAESVKGRFIISRDNSKNTLYLQMNSLRAEDTAVYYCARDRGSVEMATIADYWGQGTLVTVSS

SEQ ID NO: 23 - DNA, 366 bp, Homo sapiens
  CDS (1)..(366)
caggtccagctggtgcaatctgggggaggcgtggtccagcctgggaggtccctgagactctcctgtgcagcctctggattcagcttcagtagctatggcatgcactgggtccgccaggctccaggcaaggggctggagtgggtggcagttatatggtatgatggaagttataaatactatgcagaatccgtgaagggccgattcatcatctccagagacaattccaagaacaccctgtatctgcaaatgaacagcctgagagccgaggacacggctgtctattactgtgcgagagatcgggggtcggtagagatggctacaatcgcggactactggggccagggaaccctggtcaccgtctcctca

SEQ ID NO: 24 - protein, 122 aa, Homo sapiens; identical to SEQ ID NO: 22

SEQ ID NO: 25 - DNA, 370 bp, Homo sapiens
  CDS (1)..(369)
caggtacagctgcagcagtcaggtccaggactggtgaagccctcgcagaccctctcactcacctgtgccatctccggggacagtgtctctagcaacagtgctgcttggcactggatcaggcagtccccatcgagaggccttgagtggctgggaaggacatactacaggtccaagtggtataatgattatacagtgtctgtgaaaagtcgaataaccatcaagccagacacatccaagaaccagttctccctgcagctgaactctgtgactcccgaggacacggctgtgtattactgtgcaagatcacaggaagagcaccggtcgttggatgatgcttttgatatctgggaccacggtcaccgtctcctca

SEQ ID NO: 26 - protein, 123 aa, Homo sapiens
QVQLQQSGPGLVKPSQTLSLTCAISGDSVSSNSAAWHWIRQSPSRGLEWLGRTYYRSKWYNDYTVSVKSRITIKPDTSKNQFSLQLNSVTPEDTAVYYCARSQEEHRSLDDAFDIWDHGHRLL

SEQ ID NO: 27 - DNA, 360 bp, Homo sapiens; identical to SEQ ID NO: 15
  CDS (1)..(360)

SEQ ID NO: 28 - protein, 120 aa, Homo sapiens; identical to SEQ ID NO: 16

SEQ ID NO: 29 - DNA, 348 bp, Homo sapiens
  CDS (1)..(348)
caggtccagctggtgcaatctggggctgaggtgaggaagcccgggacgacagtgacaatctcctgcaaggtttctggacacaacttcatcgaccactacatgcattgggtacaacaggcccctggaaaagggcttgactggatgggactaattgaccctgaagatggtcagacgaaatattcagagaggtttgagggcagagtcacaattaccgcggacaagtcaacagacacaacctacttggaggtgagcggcctgagatcggaagacacggccgtttatttctgtacaacggacttgggtgacttgaattattggggccagggaaccctggtcaccgtctcctca

SEQ ID NO: 30 - protein, 116 aa, Homo sapiens
QVQLVQSGAEVRKPGTTVTISCKVSGHNFIDHYMHWVQQAPGKGLDWMGLIDPEDGQTKYSERFEGRVTITADKSTDTTYLEVSGLRSEDTAVYFCTTDLGDLNYWGQGTLVTVSS

SEQ ID NO: 31 - DNA, 366 bp, Homo sapiens
  CDS (1)..(366)
caggtccagctggtgcaatctgggggaggcgtggtccagcctgggaggtccctgagactctcctgtgcagcctctggattcaccttcagtagcaatggcatgcactgggtccgccaggctccaggcaaggggctggagtgggtggcagttatatggtatgatggaagtaataaatactatgcagactccgtgaagggccgattcaccatctccagagacaattccaagaacacactgtatctgcaaatgaacagcctgagagccgaggacacggctgtgtattactgtgcgagagatcacggccttggtgatcaagcctcctggttcgacccctggggccagggcaccctggtcaccgtctcctca

SEQ ID NO: 32 - protein, 122 aa, Homo sapiens; identical to SEQ ID NO: 4

SEQ ID NO: 33 - DNA, 368 bp, Homo sapiens
  CDS (1)..(366)
caggtccagctggtgcaatctggggctgaggtgaagaagtctggggcctcagtgaaggtctcctgcaaggcttctggatacaccttcaccggccactttatccactgggtgcggcaggcccctggacaagggcttgagtggatgggatggatcaaccctaacgttggtgtcacaaattatgcacagaagtttcagggcagggtcaccatgaccagggacacgtccataagcacagcctacatagaactgaggaggctgagatctgacgacacggccgtgtattactgtgtgagagaatccgacacagctgcggtggcctactactaccacggtatggacgtctggggacaatggtcaccgtctcttca

SEQ ID NO: 34 - protein, 122 aa, Homo sapiens
QVQLVQSGAEVKKSGASVKVSCKASGYTFTGHFIHWVRQAPGQGLEWMGWINPNVGVTNYAQKFQGRVTMTRDTSISTAYIELRRLRSDDTAVYYCVRESDTAAVAYYYHGMDVWGQWSPSL

SEQ ID NO: 35 - DNA, 375 bp, Homo sapiens
  CDS (1)..(375)
caggtccagctggtgcaatctgggggagactgggtaaagcctggggggtcccttagactctcctgtgcagcgtctggattccctttcgctaatgcctggatgtattggttccgccaggctccagggaaggggctggagtgggttggccgtattaaaagcaaaccaagtggtggggctacagagttcgctgcacccgtggaaggtagattcagcatctccagagacgattcgaaaaacacgatggatctgcaaatgaatagcctgagaaccgacgacacagccgtatattattgtaccacagattggggttcggggacctatcataagtttgctttagatgtctggggccaagggacaatggtcaccgtctcttca

SEQ ID NO: 36 - protein, 125 aa, Homo sapiens
QVQLVQSGGDWVKPGGSLRLSCAASGFPFANAWMYWFRQAPGKGLEWVGRIKSKPSGGATEFAAPVEGRFSISRDDSKNTMDLQMNSLRTDDTAVYYCTTDWGSGTYHKFALDVWGQGTMVTVSS

SEQ ID NO: 37 - DNA, 357 bp, Homo sapiens
  CDS (1)..(357)
gtgcagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggacaatggtcaccgtctcttca

SEQ ID NO: 38 - protein, 119 aa, Homo sapiens
VQLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGTMVTVSS

SEQ ID NO: 39 - DNA, 360 bp, Homo sapiens
  CDS (1)..(360)
caggtgcagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggacaatggtcaccgtctcttca

SEQ ID NO: 40 - protein, 120 aa, Homo sapiens; identical to SEQ ID NO: 16

SEQ ID NO: 41 - DNA, 360 bp, Homo sapiens
  CDS (1)..(360)
caggtccagctggtgcaatctggggctgaggcgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggaccacggtcaccgtctcctca

SEQ ID NO: 42 - protein, 120 aa, Homo sapiens
QVQLVQSGAEAKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGTTVTVSS

SEQ ID NO: 43 - DNA, 369 bp, Homo sapiens
  CDS (1)..(369)
caggtccagctggtgcaatctgggggaggcttggtacagccagggcggtccctgagactctcctgtacaacttctggattcacctttagtgattatgctttgagctgggtccgccaggctccagggagggggctggagtgggtaggtttcattagaaataaaatttatggtgggacaacagattacgccgcatctgtgaaaggcagattcaccatctcaagagatgattccaaaagtatcgcctatctgcaaatgaacagcctgaaaaccgaggactcagccgtctattactgtactagagattcgggtgtggtgactgctgcctactttgactactggggccagggcaccctggtcaccgtctcctca

SEQ ID NO: 44 - protein, 123 aa, Homo sapiens
QVQLVQSGGGLVQPGRSLRLSCTTSGFTFSDYALSWVRQAPGRGLEWVGFIRNKIYGGTTDYAASVKGRFTISRDDSKSIAYLQMNSLKTEDSAVYYCTRDSGVVTAAYFDYWGQGTLVTVSS

SEQ ID NO: 45 - DNA, 366 bp, Homo sapiens
  CDS (1)..(366)
caggtccagctggtgcaatctgggggaggcgtggtccagcctgggaggtccctgagactctcctgtgcagcctctggattcaccttcagtagcaatggcatgcactgggtccgccaggctccaggcaaggggctggagtgggtggcagttatatggtatgatggaagtaataaatactatgcagactccgtgaagggccgattcaccatctccagagacaattccaagaacacactgtatctgcaaatgaacagcctgagagccgaggacacggctgtgtattactgtgcgagagatcacggccttggtgatcaagcctcctggttcgacccctggggccaggggaccacggtcaccgtctcctca

SEQ ID NO: 46 - protein, 122 aa, Homo sapiens
QVQLVQSGGGVVQPGRSLRLSCAASGFTFSSNGMHWVRQAPGKGLEWVAVIWYDGSNKYYADSVKGRFTISRDNSKNTLYLQMNSLRAEDTAVYYCARDHGLGDQASWFDPWGQGTTVTVSS

SEQ ID NO: 47 - DNA, 360 bp, Homo sapiens; identical to SEQ ID NO: 15
  CDS (1)..(360)

SEQ ID NO: 48 - protein, 120 aa, Homo sapiens; identical to SEQ ID NO: 16

SEQ ID NO: 49 - DNA, 353 bp, Homo sapiens
  CDS (1)..(351)
cagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaagggacaatggtcaccgtctcttc

SEQ ID NO: 50 - protein, 117 aa, Homo sapiens
QLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGTMVTVS

SEQ ID NO: 51 - DNA, 360 bp, Homo sapiens
  CDS (1)..(360)
caggtccagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtctggggccaaggaaccctggtcaccgtctcttca

SEQ ID NO: 52 - protein, 120 aa, Homo sapiens
QVQLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVWGQGTLVTVSS

SEQ ID NO: 53 - DNA, 357 bp, Homo sapiens
  CDS (1)..(357)
gtccagctggtgcaatctggggctgaggtgaagaagcctggggcctcagtgaaggtctcctgtcaggcttctggatacatgttcaccggcttctatatgcactgggtgcgacaggcccctggacaagggcttgagtggatgggatggatgaacactaacagtggtgccacaggctatgcacacaagtttcaggacagggtcaccctgaccagggacacgtccatcagcacaggctacatggagctgggcggcctgacatctgacgacacggccgtgtattattgtgcgagaacccaggaggtttactactacgctatggacgtactggggccaagggacaatggtcaccgtctcttc

SEQ ID NO: 54 - protein, 119 aa, Homo sapiens
VQLVQSGAEVKKPGASVKVSCQASGYMFTGFYMHWVRQAPGQGLEWMGWMNTNSGATGYAHKFQDRVTLTRDTSISTGYMELGGLTSDDTAVYYCARTQEVYYYAMDVLGPRDNGHRLF

SEQ ID NO: 55 - DNA, 342 bp, Homo sapiens
  CDS (1)..(342)
gatattgtgatgacccagactccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaggtggaaatcaaacgt

SEQ ID NO: 56 - protein, 114 aa, Homo sapiens
DIVMTQTPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFGQGTKVEIKR

SEQ ID NO: 57 - DNA, 337 bp, Homo sapiens
  CDS (1)..(336)
gatcttgtgatgactcagtctccagactccctggctgtgtctctgggcgagagggccaccatcaactgcaagtccagccagagtgttttatacagctccaacaataagaactacttagcttggcaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaagtggatatca

SEQ ID NO: 58 - protein, 112 aa, Homo sapiens
DLVMTQSPDSLAVSLGERATINCKSSQSVLYSSNNKNYLAWHQQKPGQPPKLLIYWASTRESGVPDRFSGSGSGTDFTLTISTLQAEDVAVYYCQQYYSTPPTFGQGTKVDI

SEQ ID NO: 59 - DNA, 342 bp, Homo sapiens
  CDS (1)..(342)
gatattgtgatgactcagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaggtggaaatcaaacgt

SEQ ID NO: 60 - protein, 114 aa, Homo sapiens
DIVMTQSPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFGQGTKVEIKR

SEQ ID NO: 61 - DNA, 342 bp, Homo sapiens
  CDS (1)..(342)
gatattgtgatgactcagtctccactctccctgcccgtcacccctggagagccggcctccatctcctgcaggtctagtcagagcctcttggatagtgatgatggaaacacctatttggactggtacctgcagaagccagggcagtctccacagctcctaatctatacgctttcctatcgggcctctggagtcccagacaggttcagtggcagtgggtcaggcactgatttcacactgaaaatcagcagggtggaggctgaggatgttggagtttattactgcatgcaacgtatagagtttccttacacttttggccaggggaccaaagtggatatcaaacgt

SEQ ID NO: 62 - protein, 114 aa, Homo sapiens
DIVMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPYTFGQGTKVDIKR

SEQ ID NO: 63 - DNA, 342 bp, Homo sapiens
  CDS (1)..(342)
gatgttgtgatgactcagtctccactctccctgcccgtcacccctggagagccggcctccatctcctgcaggtctagtcagagcctcttggatagtgatgatggaaacacctatttggactggtacctgcagaagccagggcagtctccacagctcctaatctatacgctttcctatcgggcctctggagtcccagacaggttcagtggcagtgggtcaggcactgatttcacactgaaaatcagcagggtggaggctgaggatgttggagtttattactgcatgcaacgtatagagtttccttacacttttggccaggggaccaaggtggaaatcaaacgt

SEQ ID NO: 64 - protein, 114 aa, Homo sapiens
DVVMTQSPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPYTFGQGTKVEIKR

SEQ ID NO: 65 - DNA, 339 bp, Homo sapiens
  CDS (1)..(339)
gatattgtgatgacccagactccactctccctgcccgtcacccctggagagccggcctccatctcctgcaggtctagtcagagcctcttggatagtgatgatggaaacacctatttggactggtacctgcagaagccagggcagtctccacagctcctaatctatacgctttcctatcgggcctctggagtcccagacaggttcagtggcagtgggtcaggcactgatttcacactgaaaatcagcagggtggaggctgaggatgttggagtttattactgcatgcaagctacacaattgtacacttttggccaggggaccaaggtggagatcaaacgt

SEQ ID NO: 66 - protein, 113 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQATQLYTFGQGTKVEIKR

SEQ ID NO: 67 - DNA, 342 bp, Homo sapiens
  CDS (1)..(342)
gatattgtgatgactcagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaagctggagatcaaacgt

SEQ ID NO: 68 - protein, 114 aa, Homo sapiens
DIVMTQSPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFGQGTKLEIKR

SEQ ID NO: 69 - DNA, 342 bp, Homo sapiens
  CDS (1)..(342)
gatgttgtgatgactcagactccagactccctggctgtgtctctgggcgagagggccaccatcaactgcaagtccagccagagtgttttacacaagtccaacaataagaactatttagcttggtaccagcagaaaccaggacagcctcctaaattgctcattcactgggcttctacccgggaattcggggtccctgaccgactcagtggcagcgggtctgcgacagatttcactctcaccatcagcagcctgcaggctgaagacgtggcagtctattactgtcagcaatattatgctgttcctctcaccttcggccaagggacacgactggagattaaacgt

SEQ ID NO: 70 - protein, 114 aa, Homo sapiens
DVVMTQTPDSLAVSLGERATINCKSSQSVLHKSNNKNYLAWYQQKPGQPPKLLIHWASTREFGVPDRLSGSGSATDFTLTISSLQAEDVAVYYCQQYYAVPLTFGQGTRLEIKR

SEQ ID NO: 71 - DNA, 342 bp, Homo sapiens
  CDS (1)..(342)
gatattgtgatgacccagacgccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcagccaagggaccaaggtggaaatcaaacgt

SEQ ID NO: 72 - protein, 114 aa, Homo sapiens
DIVMTQTPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFSQGTKVEIKR

SEQ ID NO: 73 - DNA, 342 bp, Homo sapiens
  CDS (1)..(342)
gatgttgtgatgactcagtctccagactccctgactgtgtctctgggcgagagggccaccatcaactgcaagtccagccagagtgttttatacagctccaacaataagaactacttagcttggtaccagcagaaaccaggacagcctcctaagctgctcatttactgggcacctacccgggaatccggggtccctgaccgattcagtggcagcgggtctgggacagatttcactctcaccatcagcagcctgcaggctgaagatgtggcagtttattactgtcagcaatattatagtactcctccgacgttcggccaggggaccaaggtggaaatcaaacgt

SEQ ID NO: 74 - protein, 114 aa, Homo sapiens
DVVMTQSPDSLTVSLGERATINCKSSQSVLYSSNNKNYLAWYQQKPGQPPKLLIYWAPTRESGVPDRFSGSGSGTDFTLTISSLQAEDVAVYYCQQYYSTPPTFGQGTKVEIKR

SEQ ID NO: 75 - DNA, 342 bp, Homo sapiens
  CDS (1)..(342)
gatgttgtgatgactcagtctccagactccctggctgtgtctctgggcgagagggccaccatcaactgcaagtccagccagggtgttttacacaagtccaacaataagaactatttagcttggtaccagcagaaaccaggacagcctcctaaattgctcattcactgggcttctacccgggaattcggggtccctgaccgactcagtggcagcgggtctgcgacagatttcactctcaccatcagcagcctgcaggctgaagacgtggcagtctattactgtcagcaatattatgctgttcctctcaccttcggccaagggacacgactggagattaaacgt

SEQ ID NO: 76 - protein, 114 aa, Homo sapiens
DVVMTQSPDSLAVSLGERATINCKSSQGVLHKSNNKNYLAWYQQKPGQPPKLLIHWASTREFGVPDRLSGSGSATDFTLTISSLQAEDVAVYYCQQYYAVPLTFGQGTRLEIKR

SEQ ID NO: 77 - DNA, 342 bp, Homo sapiens
  CDS (1)..(342)
gatattgtgatgacccagacgccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaggtggaaatcaaacgt

SEQ ID NO: 78 - protein, 114 aa, Homo sapiens; identical to SEQ ID NO: 56

SEQ ID NO: 79 - DNA, 342 bp, Homo sapiens
  CDS (1)..(342)
gatgttgtgatgactcagtctccagactccctggctgtgcctctgggcgagagggccaccatcaactgcaagtccagccagagtgttttacacaagtccaacaataagaaccatttagcttggtaccagcagaaaccaggacagcctcctaaattgctcattcactgggcttctacccgggaattcggggtccctgaccgactcagtggcagcgggtctgcgacagatttcactctcaccatcaacagcctgcaggctgaagacgcggcagtctattactgtcagcaatattatgctgttcctctcaccttcggccaagggacacgactggagattaaacgt

SEQ ID NO: 80 - protein, 114 aa, Homo sapiens
DVVMTQSPDSLAVPLGERATINCKSSQSVLHKSNNKNHLAWYQQKPGQPPKLLIHWASTREFGVPDRLSGSGSATDFTLTINSLQAEDAAVYYCQQYYAVPLTFGQGTRLEIKR

SEQ ID NO: 81 - DNA, 342 bp, Homo sapiens
  CDS (1)..(342)
gatattgtgatgactcagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaagtggatatcaaacgt

SEQ ID NO: 82 - protein, 114 aa, Homo sapiens
DIVMTQSPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFGQGTKVDIKR

SEQ ID NO: 83 - DNA, 342 bp, Homo sapiens
  CDS (1)..(342)
gatgttgtgatgactcagtctccagactccctggctgtgtctctgggcgagagggccaccatcaactgcaagtccagccagagtgttttatacagctccaacaataagaactacttagcttggtaccagcagaaaccaggacagcctcctaaattgctcattcactgggcttctacccgggaattcggggtccctgaccgactcagtggcagcgggtctgcgacagatttcactctcaccatcagcagcctgcaggctgaagacgtggcagtctattactgtcagcaatattatgctgttcctctcaccttcggccaagggacacgactggagattaaacgt

SEQ ID NO: 84 - protein, 114 aa, Homo sapiens
DVVMTQSPDSLAVSLGERATINCKSSQSVLYSSNNKNYLAWYQQKPGQPPKLLIHWASTREFGVPDRLSGSGSATDFTLTISSLQAEDVAVYYCQQYYAVPLTFGQGTRLEIKR

SEQ ID NO: 85 - DNA, 342 bp, Homo sapiens
  CDS (1)..(342)
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaggtggaaatcaaacgt

SEQ ID NO: 86 - protein, 114 aa, Homo sapiens; identical to SEQ ID NO: 60

SEQ ID NO: 87 - DNA, 327 bp, Homo sapiens
  CDS (1)..(327)
gaaattgtgctgactcagtctccaggcaccctgtctttgtctccaggggaaagagccaccctctcctgcaaggccagtcagagttttagcagcaactacttagcctggtaccagcagaaacctggccaggctcccaggctgctcatctatggtgcatccagcagggccactggcatcccagacaggttcagtggcagtaaatctgggacagacttcactctcaccatcagcagactggagcctgaagattttgcagtgtattactgtcagcagtatgttacctcaccgtacacttttggcctggggaccaaggtggagatcaaacgt

SEQ ID NO: 88 - protein, 109 aa, Homo sapiens
EIVLTQSPGTLSLSPGERATLSCKASQSFSSNYLAWYQQKPGQAPRLLIYGASSRATGIPDRFSGSKSGTDFTLTISRLEPEDFAVYYCQQYVTSPYTFGLGTKVEIKR

SEQ ID NO: 89 - DNA, 325 bp, Homo sapiens
  CDS (1)..(324)
gatgttgggatgacacagtcttcagccaccctatctttgtctccaggggaaagagccaccctctcctgcagggccagtcagaggattagcagttatttagcctggtaccaacagaaacctggccaggctcccagactcctcatctatgaggcagtcaaaagggccactggcatcccagccaggttcagtggcagtgggtctgggacagagttcaccctcaccatcaacagcctagagcctgaagattttgcagtttatttctgtcagcagcgtggcagctgtcctgggacgttcggccaggggaccaagctggagatcaaacgtt

SEQ ID NO: 90 - protein, 108 aa, Homo sapiens
DVGMTQSSATLSLSPGERATLSCRASQRISSYLAWYQQKPGQAPRLLIYEAVKRATGIPARFSGSGSGTEFTLTINSLEPEDFAVYFCQQRGSCPGTFGQGTKLEIKR

SEQ ID NO: 91 - DNA, 366 bp, Homo sapiens
  CDS (1)..(366)
caggtccagctggtgcaatctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaagtatgcacagaagtttcagggcagggtcaccatgaccagggacacgtccatcagcacagcctacatggagctgagcaggctgagatctgacgacacggccgtgtattactgtgcgagaggatacgatattttgactggttatggctggttcgacccctggggccagggaaccctggtcaccgtctcctca

SEQ ID NO: 92 - protein, 122 aa, Homo sapiens
QVQLVQSGAEVKKPGASVKVSCKASGYTFTGYYMHWVRQAPGQGLEWMGWINPNSGGTKYAQKFQGRVTMTRDTSISTAYMELSRLRSDDTAVYYCARGYDILTGYGWFDPWGQGTLVTVSS

SEQ ID NO: 93 - DNA, 360 bp, Homo sapiens
  CDS (1)..(360)
caggtccagctggtgcaatctgggggaggcttggtccagcctggggggtccctgagactctcctgtgcagcctctggattcacctttagtagctattggatgagttgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacaccctgagagccgaggacacggctgtgtattactgtgcgagagatcgtttgtggacccaggggttttttgactactggggccagggaaccctggtcaccgtctcctca

SEQ ID NO: 94 - protein, 120 aa, Homo sapiens
QVQLVQSGGGLVQPGGSLRLSCAASGFTFSSYWMSWVRQAPGKGLEWVANIKQDGSEKYYVDSVKGRFTISRDNAKNSLYLQMNTLRAEDTAVYYCARDRLWTQGFFDYWGQGTLVTVSS

SEQ ID NO: 95 - DNA, 339 bp, Homo sapiens
  CDS (1)..(339)
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaacctgctcatttactgggcatctacccgggaatccggggtccctgaccgattcagtggcagcgggtctgggacagatttcactctcaccatcagcagcctgcaggctgaagatgtggcagtttattactgtcagcaatattatactactccgtggacgttcggccaagggaccaaggtggaaatcaaa

SEQ ID NO: 96 - protein, 113 aa, Homo sapiens
DIVMTQSPDSLAVSLGERATINCKSSQSVLYSSNNKNYLAWYQQKPGQPPNLLIYWASTRESGVPDRFSGSGSGTDFTLTISSLQAEDVAVYYCQQYYTTPWTFGQGTKVEIK

SEQ ID NO: 97 - DNA, 23 bp, Artificial
  Artificially Synthesized Primer Sequence
caggtkcagctggtgcagtctgg

SEQ ID NO: 98 - DNA, 23 bp, Artificial
  Artificially Synthesized Primer Sequence
caggtccagcttgtgcagtctgg

SEQ ID NO: 99 - DNA, 23 bp, Artificial
  Artificially Synthesized Primer Sequence
saggtccagctggtacagtctgg

SEQ ID NO: 100 - DNA, 23 bp, Artificial
  Artificially Synthesized Primer Sequence
caratgcagctggtgcagtctgg

SEQ ID NO: 101 - DNA, 24 bp, Artificial
  Artificially Synthesized Primer Sequence
cagatcaccttgaaggagtctggt

SEQ ID NO: 102 - DNA, 24 bp, Artificial
  Artificially Synthesized Primer Sequence
caggtcaccttgarggagtctggt

SEQ ID NO: 103 - DNA, 23 bp, Artificial
  Artificially Synthesized Primer Sequence
gargtgcagctggtggagtctgg

SEQ ID NO: 104 - DNA, 23 bp, Artificial
  Artificially Synthesized Primer Sequence
caggtgcagctggtggagtctgg

SEQ ID NO: 105 - DNA, 23 bp, Artificial
  Artificially Synthesized Primer Sequence
gaggtgcagctgttggagtctgg

SEQ ID NO: 106 - DNA, 24 bp, Artificial
  Artificially Synthesized Primer Sequence
cagstgcagctgcaggagtcgggc

SEQ ID NO: 107 - DNA, 24 bp, Artificial
  Artificially Synthesized Primer Sequence
caggtgcagctacagcagtggggc

SEQ ID NO: 108 - DNA, 24 bp, Artificial
  Artificially Synthesized Primer Sequence
gargtgcagctggtgcagtctgga

SEQ ID NO: 109 - DNA, 24 bp, Artificial
  Artificially Synthesized Primer Sequence
caggtacagctgcagcagtcaggt

SEQ ID NO: 110 - DNA, 23 bp, Artificial
  Artificially Synthesized Primer Sequence
caggtscagctggtgcaatctgg

SEQ ID NO: 111 - DNA, 24 bp, Artificial
  Artificially Synthesized Primer Sequence
tgaggagacggtgaccagggtkcc

SEQ ID NO: 112 - DNA, 24 bp, Artificial
  Artificially Synthesized Primer Sequence
tgaagagacggtgaccattgtccc

SEQ ID NO: 113 - DNA, 24 bp, Artificial
  Artificially Synthesized Primer Sequence
tgaggagacggtgaccgtggtccc

SEQ ID NO: 114 - DNA, 24 bp, Artificial
  Artificially Synthesized Primer Sequence
racatccagatgacccagtctcca

SEQ ID NO: 115 - DNA, 24 bp, Artificial
  Artificially Synthesized Primer Sequence
gmcatccagttgacccagtctcca

SEQ ID NO: 116 - DNA, 24 bp, Artificial
  Artificially Synthesized Primer Sequence
gccatccrgatgacccagtctcca

SEQ ID NO: 117 - DNA, 24 bp, Artificial
  Artificially Synthesized Primer Sequence
gtcatctggatgacccagtctcca

SEQ ID NO: 118 - DNA, 24 bp, Artificial
  Artificially Synthesized Primer Sequence
gatattgtgatgacccagactcca

SEQ ID NO: 119 - DNA, 24 bp, Artificial
  Artificially Synthesized Primer Sequence
gatrttgtgatgactcagtctcca

SEQ ID NO: 120 - DNA, 24 bp, Artificial
  Artificially Synthesized Primer Sequence
gaaattgtgttgacrcagtctcca

SEQ ID NO: 121 - DNA, 24 bp, Artificial
  Artificially Synthesized Primer Sequence
gaaatagtgatgacgcagtctcca

SEQ ID NO: 122 - DNA, 24 bp, Artificial
  Artificially Synthesized Primer Sequence
gaaattgtaatgacacagtctcca

SEQ ID NO: 123 - DNA, 24 bp, Artificial
  Artificially Synthesized Primer Sequence
gacatcgtgatgacccagtctcca

SEQ ID NO: 124 - DNA, 24 bp, Artificial
  Artificially Synthesized Primer Sequence
gaaacgacactcacgcagtctcca

SEQ ID NO: 125 - DNA, 24 bp, Artificial
  Artificially Synthesized Primer Sequence
gaaattgtgctgactcagtctcca

SEQ ID NO: 126 - DNA, 24 bp, Artificial
  Artificially Synthesized Primer Sequence
gatgttgtgatgacacagtctcca

SEQ ID NO: 127 - DNA, 24 bp, Artificial
  Artificially Synthesized Primer Sequence
acgtttgatttccaccttggtccc

SEQ ID NO: 128 - DNA, 24 bp, Artificial
  Artificially Synthesized Primer Sequence
acgtttgatctccascttggtccc

SEQ ID NO: 129 - DNA, 24 bp, Artificial
  Artificially Synthesized Primer Sequence
acgtttgatatccactttggtccc

SEQ ID NO: 130 - DNA, 24 bp, Artificial
  Artificially Synthesized Primer Sequence
acgtttaatctccagtcgtgtccc

SEQ ID NO: 131 - DNA, 24 bp, Artificial
  Artificially Synthesized Primer Sequence
cagtctgtgctgactcagccaccc

SEQ ID NO: 132 - DNA, 24 bp, Artificial
  Artificially Synthesized Primer Sequence
cagtctgtgytgacgcagccgccc

SEQ ID NO: 133 - DNA, 22 bp, Artificial
  Artificially Synthesized Primer Sequence
cagtctgccctgactcagccts

SEQ ID NO: 134 - DNA, 24 bp, Artificial
  Artificially Synthesized Primer Sequence
tcctatgwgctgactcagccaccc

SEQ ID NO: 135 - DNA, 24 bp, Artificial
  Artificially Synthesized Primer Sequence
tcctatgagctgacacagcyaccc

SEQ ID NO: 136 - DNA, 24 bp, Artificial
  Artificially Synthesized Primer Sequence
tcttctgagctgactcaggaccct

SEQ ID NO: 137 - DNA, 24 bp, Artificial
  Artificially Synthesized Primer Sequence
tcctatgagctgatgcagccaccc

SEQ ID NO: 138 - DNA, 24 bp, Artificial
  Artificially Synthesized Primer Sequence
cagcctgtgctgactcaatcatcc

SEQ ID NO: 139 - DNA, 24 bp, Artificial
  Artificially Synthesized Primer Sequence
cagcttgtgctgactcaatcgccc

SEQ ID NO: 140 - DNA, 24 bp, Artificial
  Artificially Synthesized Primer Sequence
ctgcctgtgctgactcagcccccg

SEQ ID NO: 141 - DNA, 24 bp, Artificial
  Artificially Synthesized Primer Sequence
cagcctgtgctgactcagccayct

SEQ ID NO: 142 - DNA, 24 bp, Artificial
  Artificially Synthesized Primer Sequence
caggctgtgctgactcagccggct

SEQ ID NO: 143 - DNA, 24 bp, Artificial
  Artificially Synthesized Primer Sequence
aattttatgctgactcagccccac

SEQ ID NO: 144 - DNA, 24 bp, Artificial
  Artificially Synthesized Primer Sequence
cagrctgtggtgactcaggagccc

SEQ ID NO: 145 - DNA, 24 bp, Artificial
  Artificially Synthesized Primer Sequence
cagactgtggtgacccaggagcca

SEQ ID NO: 146 - DNA, 24 bp, Artificial
  Artificially Synthesized Primer Sequence
cwgcctgtgctgactcagccacct

SEQ ID NO: 147 - DNA, 24 bp, Artificial
  Artificially Synthesized Primer Sequence
caggcagggctgactcagccaccc

SEQ ID NO: 148 - DNA, 24 bp, Artificial
  Artificially Synthesized Primer Sequence
acctaggacggtgaccttggtccc

SEQ ID NO: 149 - DNA, 24 bp, Artificial
  Artificially Synthesized Primer Sequence
acctaggacggtcagcttggtccc

SEQ ID NO: 150 - DNA, 24 bp, Artificial
  Artificially Synthesized Primer Sequence
accgaggacggtcagctgggtgcc

SEQ ID NO: 151 - DNA, 91 bp, Artificial
  Template Linker Sequence
ggacaatggtcaccgtctcttcaggtggtggtggttcgggtggtggtggttcgggtggtggcggatcggacatccagatgacccagtctcc

SEQ ID NO: 152 - DNA, 28 bp, Artificial
  Artificially Synthesized Primer Sequence
gcaccctggtcaccgtctcctcaggtgg

SEQ ID NO: 153 - DNA, 28 bp, Artificial
  Artificially Synthesized Primer Sequence
ggacaatggtcaccgtctcttcaggtgg

SEQ ID NO: 154 - DNA, 28 bp, Artificial
  Artificially Synthesized Primer Sequence
gaaccctggtcaccgtctcctcaggtgg

SEQ ID NO: 155 - DNA, 28 bp, Artificial
  Artificially Synthesized Primer Sequence
ggaccacggtcaccgtctcctcaggtgg

SEQ ID NO: 156 - DNA, 32 bp, Artificial
  Artificially Synthesized Primer Sequence
ggagactgggtcatctggatgtccgatccgcc

SEQ ID NO: 157 - DNA, 32 bp, Artificial
  Artificially Synthesized Primer Sequence
ggagactgagtcatcacaacatccgatccgcc

SEQ ID NO: 158 - DNA, 32 bp, Artificial
  Artificially Synthesized Primer Sequence
ggagactgcgtcaacacaatttccgatccgcc

SEQ ID NO: 159 - DNA, 32 bp, Artificial
  Artificially Synthesized Primer Sequence
ggagactgggtcatcacgatgtccgatccgcc

SEQ ID NO: 160 - DNA, 32 bp, Artificial
  Artificially Synthesized Primer Sequence
ggagactgcgtgagtgtcgtttccgatccgcc

SEQ ID NO: 161 - DNA, 32 bp, Artificial
  Artificially Synthesized Primer Sequence
ggagactgagtcagcacaatttccgatccgcc

SEQ ID NO: 162 - DNA, 42 bp, Artificial
  Artificially Synthesized Primer Sequence
ggcggctgcgtcaacacagactgcgatccgccaccgccagag

SEQ ID NO: 163 - DNA, 42 bp, Artificial
  Artificially Synthesized Primer Sequence
gcaggctgagtcagagcagactgcgatccgccaccgccagag

SEQ ID NO: 164 - DNA, 42 bp, Artificial
  Artificially Synthesized Primer Sequence
ggtggctgagtcagcacataggacgatccgccaccgccagag

SEQ ID NO: 165 - DNA, 42 bp, Artificial
  Artificially Synthesized Primer Sequence
gggtcctgagtcagctcagaagacgatccgccaccgccagag

SEQ ID NO: 166 - DNA, 42 bp, Artificial
  Artificially Synthesized Primer Sequence
ggcggttgagtcagtataacgtgcgatccgccaccgccagag

SEQ ID NO: 167 - DNA, 42 bp, Artificial
  Artificially Synthesized Primer Sequence
gacggctgagtcagcacagactgcgatccgccaccgccagag

SEQ ID NO: 168 - DNA, 42 bp, Artificial
  Artificially Synthesized Primer Sequence
tggggctgagtcagcataaaattcgatccgccaccgccagag

SEQ ID NO: 169 - DNA, 39 bp, Artificial
  Artificially Synthesized Primer Sequence
agtattgaccatggcccaggtgcagctggtgcagtctgg

SEQ ID NO: 170 - DNA, 39 bp, Artificial
  Artificially Synthesized Primer Sequence
agtattgaccatggcccaggtcaacttaagggagtctgg

SEQ ID NO: 171 - DNA, 39 bp, Artificial
  Artificially Synthesized Primer Sequence
agtattgaccatggccgaggtgcagctggtggagtctgg

SEQ ID NO: 172 - DNA, 39 bp, Artificial
  Artificially Synthesized Primer Sequence
agtattgaccatggcccaggtgcagctgcaggagtcggg

SEQ ID NO: 173 - DNA, 39 bp, Artificial
  Artificially Synthesized Primer Sequence
agtattgaccatggcccaggtgcagctgttgcagtctgc

SEQ ID NO: 174 - DNA, 39 bp, Artificial
  Artificially Synthesized Primer Sequence
agtattgaccatggcccaggtacagctgcagcagtcagg

SEQ ID NO: 175 - DNA, 34 bp, Artificial
  Artificially Synthesized Primer Sequence
taatgaattcacgtttgatttccaccttggtccc

SEQ ID NO: 176 - DNA, 34 bp, Artificial
  Artificially Synthesized Primer Sequence
taatgaattcacgtttgatctccagcttggtccc

SEQ ID NO: 177 - DNA, 34 bp, Artificial
  Artificially Synthesized Primer Sequence
taatgaattcacgtttgatatccactttggtccc

SEQ ID NO: 178 - DNA, 34 bp, Artificial
  Artificially Synthesized Primer Sequence
taatgaattcacgtttgatctccaccttggtccc

SEQ ID NO: 179 - DNA, 34 bp, Artificial
  Artificially Synthesized Primer Sequence
taatgaattcacgtttaatctccagtcgtgtccc

SEQ ID NO: 180 - DNA, 34 bp, Artificial
  Artificially Synthesized Primer Sequence
taatgaattcacctaggacggtgaccttggtccc

SEQ ID NO: 181 - DNA, 34 bp, Artificial
  Artificially Synthesized Primer Sequence
taatgaattcacctaggacggtcagcttggtccc

SEQ ID NO: 182 - DNA, 34 bp, Artificial
  Artificially Synthesized Primer Sequence
taatgaattcacctaaaacggtgagctgggtccc

SEQ ID NO: 183 - DNA, 861 bp, Homo sapiens
  CDS (1)..(861)
atgaaatacctgctgccgaccgctgctgctggtctgctgctcctcgctgcccagccggcgatggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaagtatgcacagaagtttcagggcagggtcaccatgaccagggacacgtccatcagcacagcctacatggagctgagcaggctgagatctgacgacacggccgtgtattactgtgcgagaggatacgatattttgactggttatggctggttcgacccctggggccagggaaccctggtcaccgtctcctcaggtggtggtggttcgggtggtggtggttcgggtggtggcggatcggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaaactgctcatttactgggcatctacccgggaatccggggtccctgaccgattcagtggcagcgggtctgggacagatttcactctcaccatcagcaccctgcaggctgaagatgtggcagtttattactgtcagcaatattatagtactcctccgacgttcggccaagggaccaaggtggaaatcaaacgtcgtgaattcgactacaaggatgacgacgataagtga

SEQ ID NO: 184 - protein, 286 aa, Homo sapiens
MKYLLPTAAAGLLLLAAQPAMAMAQVQLVQSGAEVKKPGASVKVSCKASGYTFTGYYMHWVRQAPGQGLEWMGWINPNSGGTKYAQKFQGRVTMTRDTSISTAYMELSRLRSDDTAVYYCARGYDILTGYGWFDPWGQGTLVTVSSGGGGSGGGGSGGGGSDIVMTQSPDSLAVSLGERATINCKSSQSVLYSSNNKNYLAWYQQKPGQPPKLLIYWASTRESGVPDRFSGSGSGTDFTLTISTLQAEDVAVYYCQQYYSTPPTFGQGTKVEIKRREFDYKDDDDK

SEQ ID NO: 185 - DNA, 846 bp, Homo sapiens
  CDS (1)..(846)
atgaaatacctgctgccgaccgctgctgctggtctgctgctcctcgctgcccagccggcgatggccatggcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaagtatgcacagaagtttcagggcagggtcaccatgaccagggacacgtccatcagcacagcctacatggagctgagcaggctgagatctgacgacacggccgtgtattactgtgcgagaggatacgatattttgactggttatggctggttcgacccctggggccagggaaccctggtcaccgtctcctcaggtggtggtggttcgggtggtggtggttcgggtggtggcggatcggaaattgtgctgactcagtctccaggcaccctgtctttgtctccaggggaaagagccaccctctcctgcaaggccagtcagagttttagcagcaactacttagcctggtaccagcagaaacctggccaggctcccaggctgctcatctatggtgcatccagcagggccactggcatcccagacaggttcagtggcagtaaatctgggacagacttcactctcaccatcagcagactggagcctgaagattttgcagtgtattactgtcagcagtatgttacctcaccgtacacttttggccaggggaccaaggtggagatcaaacgtcgtgaattcgactacaaggatgacgacgataagtga

SEQ ID NO: 186 - protein, 281 aa, Homo sapiens
MKYLLPTAAAGLLLLAAQPAMAMAQVQLVQSGAEVKKPGASVKVSCKASGYTFTGYYMHWVRQAPGQGLEWMGWINPNSGGTKYAQKFQGRVTMTRDTSISTAYMELSRLRSDDTAVYYCARGYDILTGYGWFDPWGQGTLVTVSSGGGGSGGGGSGGGGSEIVLTQSPGTLSLSPGERATLSCKASQSFSSNYLAWYQQKPGQAPRLLIYGASSRATGIPDRFSGSKSGTDFTLTISRLEPEDFAVYYCQQYVTSPYTFGQGTKVEIKRREFDYKDDDDK

SEQ ID NO: 187 - DNA, 852 bp, Homo sapiens
  CDS (1)..(852)
atgaaatacctgctgccgaccgctgctgctggtctgctgctcctcgctgcccagccggcgatggccatggcccaggtccagctggtgcaatctgggggaggcttggtccagcctggggggtccctgagactctcctgtgcagcctctggattcacctttagtagctattggatgagttgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacaccctgagagccgaggacacggctgtgtattactgtgcgagagatcgtttgtggacccaggggttttttgactactggggccagggaaccctggtcaccgtctcctcaggtggtggtggttcgggtggtggtggttcgggtggtggcggatcggacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaacctgctcatttactgggcatctacccgggaatccggggtccctgaccgattcagtggcagcgggtctgggacagatttcactctcaccatcagcagcctgcaggctgaagatgtggcagtttattactgtcagcaatattatactactccgtggacgttcggccaagggaccaaggtggaaatcaaacgtgaattcgactacaaggatgacgacgataagtga

SEQ ID NO: 188 - protein, 283 aa, Homo sapiens
MKYLLPTAAAGLLLLAAQPAMAMAQVQLVQSGGGLVQPGGSLRLSCAASGFTFSSYWMSWVRQAPGKGLEWVANIKQDGSEKYYVDSVKGRFTISRDNAKNSLYLQMNTLRAEDTAVYYCARDRLWTQGFFDYWGQGTLVTVSSGGGGSGGGGSGGGGSDIVMTQSPDSLAVSLGERATINCKSSQSVLYSSNNKNYLAWYQQKPGQPPNLLIYWASTRESGVPDRFSGSGSGTDFTLTISSLQAEDVAVYYCQQYYTTPWTFGQGTKVEIKREFDYKDDDDK